(19) United States
(12) Patent Application Publication
(10) Pub. No.: US 2003/0092175 A1
(43) Pub. Date: May 15, 2003

(54) HUMAN PROTEINS HAVING TRANSMEMBRANE DOMAINS AND DNAS ENCODING THESE PROTEINS

(76) Inventors: SEISHI KATO, KANAGAWA (JP); SHINGO SEKINE, SAITAMA (JP); TOMOKO KIMURA, KANAGAWA (JP); MIDORI KOBAYASHI, KANAGAWA (JP)

Correspondence Address: AMY E. MANDRAGOURAS, ESQ., LAHIVE AND COCKFIELD, LLP, 28 STATE STREET, BOSTON, MA, 02109, US

(21) Appl. No.: 09/284,320

(22) PCT Filed: Nov. 7, 1997
(86) PCT No.: PCT/JP97/04056

(30) Foreign Application Priority Data
Nov. 13, 1996 (JP) 8301429

(51) Int. Cl. C12P 21/06; C12N 5/10; C12N 5/06; C07K 17/00; C07H 21/04; C07K 14/00; C07K 1/00

(52) U.S. Cl. 435/365.1; 536/23.5; 530/350; 435/69.1

(57) ABSTRACT

Proteins containing any of the amino acid sequences represented by Sequence No. 1 to Sequence No. 2 or by Sequence No. 4 to Sequence No. 25 and DNAs encoding said proteins exemplified by cDNAs containing any of the base sequences represented by Sequence No. 26 to Sequence No. 50. Said proteins can be provided by expressing cDNAs encoding human proteins having transmembrane domains and recombinants of these human cDNAs.

### TECHINICAL FIELD

The present invention relates to human proteins having transmembrane domains, DNAs encoding these proteins and eukaryotic cells expressing those DNAs. The proteins of the present invention can be used as pharmaceuticals or as antigens for preparing antibodies against said proteins. The cDNAs of the present invention can be used as probes for the gene diagnosis and gene sources for the gene therapy. Furthermore, the cDNAs can be used as gene sources for large-scale production of the proteins encoded by said cDNAs. Moreover, the cells introduced with DNAs encoding transmembrane proteins therein and expressing transmembrane proteins in large amounts can be used for detection of the corresponding ligands as well as screening of novel low molecular medicines.

### BACKGROUND ART

Membrane proteins play important roles, as signal receptors, ion channels, transporters, etc., for the material transportation and the information transmission which are mediated by the cell membrane. Their examples include receptors for a variety of cytokines, ion channels for the sodium ion, the potassium ion, the chloride ion, etc., transporters for saccharides and amino acids, and so on, where the genes for many of them have been cloned already.

It has been clarified that the abnormalities of these membrane proteins are related to a number of hitherto cryptogenic diseases. For example, a gene for a membrane protein having 12 transmembrane domains was identified as the gene responsible for cystic fibrosis [Rommens, J. M. et al., Science 245: 1059-1065 (1989)]. In addition, it has been clarified that several membrane proteins act as the receptors when a virus infects the cells. For example, HIV-1 is revealed to infect into the cells through the mediation of a membrane protein fusin, a membrane protein on the T-cell membrane, having a CD-4 antigen and 7 transmembrane domains [Feng, Y. et al., Science 272: 872-877 (1996)]. Therefore, discovery of a new membrane protein is anticipated to lead to the elucidation of the causes of many diseases, whereby isolation of a new gene coding for the membrane protein has been desired.

Heretofore, owing to difficulty in the purification, many of membrane proteins have been isolated by an approach from the gene side. A general method is the so-called expression cloning which comprises transfection of a cDNA library in the animal cells to express the cDNA and then detection of the cells expressing the target membrane protein on the membrane by an immunological technique using an antibody or a biological technique for the change in the membrane permeability. However, this method is applicable only to cloning of a gene for a membrane protein with a known function.

In general, membrane proteins possess hydrophobic transmembrane domains inside the proteins which are synthesized in the ribosome and then remain in the phospholipid to be trapped in the membrane. Accordingly, the evidence of the cDNA for encoding the membrane protein is provided by determination of the whole base sequence of a full-length cDNA followed by detection of highly hydrophobic transmembrane domains in the amino acid sequence of the protein encoded by said cDNA.

The object of the present invention is to provide novel human proteins having transmembrane domains, DNAs encoding said proteins and transformed eukaryotic cells capable of expressing said DNAs.

As the result of intensive studies, the present inventors were successful in cloning of cDNAs having transmembrane domains from a human full-length cDNA bank, thereby completing the present invention. That is to say, the present invention provides proteins containing any of the amino acid sequences represented by Sequence No. 1 to Sequence No. 2 or by Sequence No. 4 to Sequence No. 25 that are human proteins having transmembrane domains. The present invention also provides DNAs encoding said proteins such as cDNAs containing any of the base sequences represented by Sequence No. 26 to Sequence No. 50 and transformed eukaryotic cells capable of expressing said DNAs.

Each of the proteins of the present invention can be obtained, for example, by a method for isolation from human organs, cell lines, etc, a method for preparation of the peptide by the chemical synthesis on the basis of the amino acid sequence of the present invention, or a method for production with the recombinant DNA technology using the DNA encoding the transmembrane domains of the present invention, wherein the method for obtainment by the recombinant DNA technology is employed preferably. For example, an in vitro expression can be achieved by preparation of an RNA by the in vitro transcription from a vector having a cDNA of the present invention, followed by the in vitro translation using this RNA as a template. Also, the recombination of the translation domain to a suitable expression vector by the method known in the art leads to the expression of a large amount of the encoded protein by using prokaryotic cells (e.g. Escherichia coli, Bacillus subtilis) or eukaryotic cells (e.g. yeasts, insect cells, animal cells).

In the case in which a protein of the present invention is expressed by a microorganism such as Escherichia coli, the translation region of a cDNA of the present invention is constructed in an expression vector having an origin, a promoter, ribosome-binding site(s), cDNA-cloning site(s), a terminator, etc. that can be replicated in the microorganism and, after transformation of the host cells with said expression vector, the thus-obtained transformant is incubated, whereby the protein encoded by said cDNA can be produced on a large scale in the microorganism. In that case, a protein fragment containing an optional region can be obtained by performing the expression with inserting an initiation codon and a termination codon before and after the optional translation region. Alternatively, a fusion protein with another protein can be expressed. Only a protein portion encoding said cDNA can be obtained by cleavage of said fusion protein with an appropriate protease.

In the case wherein a protein of the present invention is to be produced in eukaryotic cells, the translation region of said cDNA may be subjected to recombination to an expression vector for eukaryotic cells having a promoter, a splicing domain, a poly(A) addition site, etc. and transfected into the eukaryotic cells so that the protein is produced as a membrane protein on the cell membrane surface. As the expression vector, there are exemplified pKA1, pCDM8, pSVK3, pMSG, pSVL, PBK-CMV, pBK-RSV, EBV vector, pRS, pYES2, etc. Examples of the eukaryotic cells are mamamlian animal culture cells (e.g. simian renal cells COS7, chinese hamster ovarian cells CHO), blast yeasts, fission yeasts, silkworm yeasts, South African clawed toad oocytes, etc. However, any eukaryotic cells may be used insofar as the protein of the invention can be expressed on the cell membrane surface. In order to introduce the expression vector into the eukaryotic cells, there may be used any per se conventional method such as electroporation method, calcium phosphate method, liposome method or DEAE dextran method.

For separation and purification of the protein of the invention from the culture after expression of such protein in prokaryotic cells or eukaryotic cells, conventional separation operations may be adopted, if necessary, in their proper combinaion. Examples of the conventional separation operations are treatment with a denaturing agent (e.g. urea) or a surfactant, ultrasonic treatment, enzymatic digestion, salting out, solvent precipitation, dialysis, centrifugation, ultrafiltration, gel filtration, SDS-PAGE, isoelectric point electrophoresis, ion exchange chromatography, hydrophobic chromatography, affinity chromatography, reverse phase chromatography, etc.

The proteins of the present invention include peptide fragments (more than 5 amino acid residues) containing any partial amino acid sequence of the amino acid sequences represented by Sequence No. 1 to Sequence No. 2 or by Sequence No. 4 to Sequence No. 25. These fragments can be used as antigens for preparation of the antibodies. Also, the proteins of the present invention that have signal sequences appear in the form of maturation proteins on the cell surface, after the signal sequences are removed. Therefore, these maturation proteins shall come within the scope of the present invention. The N-terminal amino acid sequences of the maturation proteins can be easily identified by using the method for the cleavage-site determination in a signal sequence [Japanese Patent Kokai Publication No. 1996-187100]. Furthermore, many membrane proteins are subjected to the processing on the cell surface to be converted to the secretor forms. These secretor proteins or peptides shall come within the scope of the present invention. When glycosylation sites are present in the amino acid sequences, expression in appropriate animal cells affords glycosylated proteins. Therefore, these glycosylated proteins or peptides also shall come within the scope of the present invention.

The DNAs of the present invention include all DNAs encoding the above-mentioned proteins. Said DNAs can be obtained using the method by chemical synthesis, the method by cDNA cloning, and so on.

Each of the cDNAs of the present invention can be cloned from, for example, a cDNA library of the human cell origin. The cDNA is synthesized using as a template a poly(A)⁺ RNA extracted from human cells. The human cells may be cells delivered from the human body, for example, by the operation or may be the culture cells. The cDNA can be synthesized by using any method selected from the Okayama-Berg method [Okayama, H. and Berg, P., Mol. Cell. Biol. 2: 161-170 (1982)], the Gubler-Hoffman method [Gubler, U. and Hoffman, J. Gene 25: 263-269 (1983)], and so on, but it is preferred to use the capping method [Kato, S. et al., Gene 150: 243-250 (1994)] as illustrated in Examples in order to obtain a full-length clone in an effective manner.

The primary selection of a cDNA encoding a human protein having transmembrane domain(s) is performed by the sequencing of a partial base sequence of the cDNA clone selected at random from the cDNA library, sequencing of the amino acid sequence encoded by the base sequence, and recognition of the presence or absence of hydrophobic site(s) in the resulting N-terminal amino acid sequence region. Next, the secondary selection is carried out by determination of the whole base sequence by the sequencing and the protein expression by the in vitro translation. The ascertainment of the cDNA of the present invention for encoding the protein having the secretory signal sequence is performed by using the signal sequence detection method [Yokoyama-Kobayashi, M. et al., Gene 163: 193-196 (1995)]. In other words, the ascertainment for the coding portion of the inserted cDNA fragment to function as a signal sequence is provided by fusing a cDNA fragment encoding the N-terminus of the target protein with a cDNA encoding the protease domain of urokinase and then expressing the resulting cDNA in COS7 cells to detect the urokinase activity in the cell culture medium. On the other hand, the N-terminal region is judged to remain in the membrane in the case where the urokinase activity is not detected in the cell culture medium.

The cDNAs of the present invention are characterized by containing any of the base sequences represented by Sequence No. 26 to Sequence No. 50 and any of the base sequences represented by Sequence No. 51 to Sequence No. 75. Table 1 summarizes the clone number (HP number), the cells affording the cDNA, the total base number of the cDNA, and the number of the amino acid residues of the encoded protein, for each of the cDNAs.

TABLE 1

| | | | | Number of |
|---|---|---|---|---|
| Sequence | | | Number | Amino Acid |
| Number | HP Number | Cells | of bases | Residues |
| 1, 26, 51 | HP00442 | HT-1080 | 986 | 205 |
| 2, 27, 52 | HP00804 | Leucocyte | 1824 | 371 |
| 3, 28, 53 | HP01098 | Stomach | 1076 | 179 |
| | | cancer | | |
| 4, 29, 54 | HP01148 | Liver | 1591 | 347 |
| 5, 30, 55 | HP01293 | Liver | 1888 | 554 |
| 6, 31, 56 | HP10013 | KB | 2033 | 350 |
| 7, 32, 57 | HP10034 | HT-1080 | 911 | 209 |
| 8, 33, 58 | HP10050 | HT-1080 | 601 | 163 |
| 9, 34, 59 | HP10071 | Stomach | 394 | 92 |
| | | cancer | | |
| 10, 35, 60 | HP10076 | U937 | 732 | 172 |
| 11, 36, 61 | HP10085 | U937 | 697 | 149 |
| 12, 37, 62 | HP10122 | Stomach | 1186 | 188 |
| | | cancer | | |
| 13, 38, 63 | HP10136 | U937 | 1409 | 215 |
| 14, 40, 64 | HP10175 | Stomach | 974 | 112 |
| | | cancer | | |
| 15, 41, 65 | HP10179 | KB | 925 | 114 |
| 16, 41, 66 | HP10196 | HT-1080 | 1115 | 327 |
| 17, 42, 67 | HP10235 | HT-1080 | 1721 | 373 |
| 18, 43, 68 | HP10297 | Stomach | 1504 | 183 |
| | | cancer | | |
| 19, 44, 69 | HP10299 | Stomach | 532 | 116 |
| | | cancer | | |
| 20, 45, 70 | HP10301 | KB | 662 | 152 |
| 21, 46, 71 | HP10302 | Liver | 2373 | 559 |
| 22, 47, 72 | HP10304 | U-2 OS | 1404 | 330 |
| 23, 48, 73 | HP10305 | U-2 OS | 893 | 108 |
| 24, 49, 74 | HP10306 | U-2 OS | 690 | 101 |
| 25, 50, 75 | HP10328 | KB | 2186 | 372 |

Hereupon, the same clone as any of the cDNAs of the present invention can be easily obtained by screening of the cDNA library constructed from the cell line or the human tissue employed in the present invention, by the use of an oligonucleotide probe synthesized on the basis of the corresponding cDNA base sequence depicted in Sequence No. 51 to Sequence No. 75.

In general, the polymorphism due to the individual difference is frequently observed in human genes. Therefore, any cDNA that is subjected to insertion or deletion of one or plural nucleotides and/or substitution with other nucleotides in Sequence No. 51 to Sequence No. 75 shall come within the scope of the present invention.

In a similar manner, any protein that is produced by these modifications comprising insertion or deletion of one or plural nucleotides and/or substitution with other nucleotides shall come within the scope of the present invention, as far as said protein possesses the activity of the corresponding protein having the amino acid sequence represented by Sequence No. 1 to Sequence No. 2 or by Sequence No. 4 to Sequence No. 25.

The cDNAs of the present invention include cDNA fragments (more than 10 bp) containing any partial base sequence of the base sequence represented by Sequence No. 26 to No. 50 or of the base sequence represented by Sequence No. 51 to No. 75. Also, DNA fragments consisting of a sense chain and an anti-sense chain shall come within this scope. These DNA fragments can be used as the probes for the gene diagnosis.

### BRIEF DESCRIPTION OF DRAWINGS

FIG. 1: A figure depicting the structure of the secretory signal sequence detection vector pSSD3.

FIG. 2: A figure depicting the hydrophobicity/hydrophilicity profile of the protein encoded by clone HP00442.

FIG. 3: A figure depicting the hydrophobicity/hydrophilicity profile of the protein encoded by clone HP00804.

FIG. 4: A figure showing the result on the northern-blot hybridization of clone HP00804.

FIG. 5: A figure depicting the hydrophobicity/hydrophilicity profile of the protein encoded by clone HP01098.

FIG. 6: A figure depicting the hydrophobicity/hydrophilicity profile of the protein encoded by clone HP01148.

FIG. 7: A figure showing the result on the northern-blot hybridization of clone HP01148.

FIG. 8: A figure depicting the hydrophobicity/hydrophilicity profile of the protein encoded by clone HP01293.

FIG. 9: A figure depicting the hydrophobicity/hydrophilicity profile of the protein encoded by clone HP10013.

FIG. 10: A figure depicting the hydrophobicity/hydrophilicity profile of the protein encoded by clone HP10034.

FIG. 11: A figure depicting the hydrophobicity/hydrophilicity profile of the protein encoded by clone HP10050.

FIG. 12: A figure depicting the hydrophobicity/hydrophilicity profile of the protein encoded by clone HP10071.

FIG. 13: A figure depicting the hydrophobicity/hydrophilicity profile of the protein encoded by clone HP10076.

FIG. 14: A figure depicting the hydrophobicity/hydrophilicity profile of the protein encoded by clone HP10085.

FIG. 15: A figure depicting the hydrophobicity/hydrophilicity profile of the protein encoded by clone HP10122.

FIG. 16: A figure depicting the hydrophobicity/hydrophilicity profile of the protein encoded by clone HP10136.

FIG. 17: A figure depicting the hydrophobicity/hydrophilicity profile of the protein encoded by clone HP10175.

FIG. 18: A figure depicting the hydrophobicity/hydrophilicity profile of the protein encoded by clone HP10179.

FIG. 19: A figure depicting the hydrophobicity/hydrophilicity profile of the protein encoded by clone HP10196.

FIG. 20: A figure depicting the hydrophobicity/hydrophilicity profile of the protein encoded by clone HP10235.

FIG. 21: A figure depicting the hydrophobicity/hydrophilicity profile of the protein encoded by clone HP10297.

FIG. 22: A figure depicting the hydrophobicity/hydrophilicity profile of the protein encoded by clone HP10299.

FIG. 23: A figure depicting the hydrophobicity/hydrophilicity profile of the protein encoded by clone HP10301.

FIG. 24: A figure depicting the hydrophobicity/hydrophilicity profile of the protein encoded by clone HP10302.

FIG. 25: A figure depicting the hydrophobicity/hydrophil the protein encoded by clone HP10304.

FIG. 26: A figure depicting the hydrophobicity/hydrophilicity profile of the protein encoded by clone HP10305.

FIG. 27: A figure depicting the hydrophobicity/hydrophilicity profile of the protein encoded by clone HP10306.

FIG. 28: A figure depicting the hydrophobicity/hydrophilicity profile of the protein encoded by clone HP10328.

### BEST MODE FOR CARRING OUT INVENTION

### EXAMPLE

The present invention is embodied in more detail by the following examples, but this embodiment is not intended to restrict the present invention. The basic operations and the enzyme reactions with regard to the DNA recombination are carried out according to the literature [Molecular Cloning. A Laboratory Manual”, Cold Spring Harbor Laboratory, 1989]. Unless otherwise stated, restrictive enzymes and a variety of modification enzymes to be used were those available from TAKARA SHUZO. The manufacturer's instructions were used for the buffer compositions as well as for the reaction conditions, in each of the enzyme reactions. The cDNA synthesis was carried out according to the literature [Kato, S. et al., Gene 150: 243-250 (1994)].

(1) Preparation of Poly(A)⁺ RNA

The fibrosarcoma cell line HT-1080 (ATCC CCL 121), the epidermoid carcinoma cell line KB (ATCC CRL 17), the histiocyte lymphoma cell line U937 (ATCC CRL 1593), the osterosarcoma U-2 OS (ATCC HTB 96), a leukocyte isolated from the peripheral blood, tissues of stomach cancer delivered by the operation, and liver were used for human cells to extract mRNAs. Each of the cell lines was cultured by a conventional procedure.

After about 1 g of human tissues was homogenized in 20 ml of a 5.5 M guanidinium thiocyanate solution, total mRNAs were prepared in accordance with the literature [Okayama, H. et al., “Methods in Enzymology” Vol. 164, Academic Press, 1987]. These mRNAs were subjected to chromatography using an oligo(dT)-cellulose column washed with 20 mM Tris-hydrochloric acid buffer solution (pH 7.6), 0.5 M NaCl, and 1 mM EDTA to obtain a poly(A)⁺ RNA in accordance with the above-mentioned literature.

(2) Construction of cDNA Library

To a solution of 10 μg of the above-mentioned poly(A)⁺ RNA in 100 mM Tris-hydrochloric acid buffer solution (pH 8) was added one unit of an RNase-free, bacterium-origin alkaline phosphatase and the resulting solution was allowed to react at 37° C. for one hour. After the reaction solution underwent the phenol extraction followed by the ethanol precipitation, the obtained pellets were dissolved in a mixed solution of 50 mM sodium acetate (pH 6), 1 mM EDTA, 0.1% 2-mercaptoethanol, and 0.01% Triton X-100. Thereto was added one unit of a tobacco-origin pyrophosphatase (Epicenter Technologies) and the resulting solution at a total volume of 100 μl was allowed to react at 37° C. for one hour. After the reaction solution underwent the phenol extraction followed by the ethanol precipitation, the thus-obtained pellets were dissolved in water to obtain a decapped poly(A)⁺ RNA solution.

To a solution of the decapped poly(A)⁺ RNA and 3 nmol of a DNA-RNA chimeric oligonucleotide (5′-dG-dG-dG-dG-dA-dA-dT-dT-dC-dG-dA-G-G-A-3′) in a mixed aqueous solution of 50 mM Tris-hydrochloric acid buffer solution (pH 7.5), 0.5 mM ATP, 5 mM MgCl₂, 10 mM 2-mercaptoethanol, and 25% polyethylene glycol were added 50 units of T4 RNA ligase and the resulting solution at a total volume of 30 μl was allowed to react at 20° C. for 12 hours. After the reaction solution underwent the phenol extraction followed by the ethanol precipitation, the thus-obtained pellets were dissolved in water to obtain a chimeric oligo-capped poly(A)⁺ RNA.

After the vector pKA1 developed by the present inventors (Japanese Patent Kokai Publication No. 1992-117292) was digested with KpnI, an about 60-dT tail was inserted by a terminal transferase. This product was digested with EcoRV to remove the dT tail at one side and the resulting molecule was used as a vectorial primer.

After 6 μg of the previously-prepared chimeric oligo-capped poly(A)⁺ RNA was annealed with 1.2 μg of the vectorial primer, the product was dissolved in a mixed solution of 50 mM Tris-hydrochloric acid buffer solution (pH 8.3), 75 mM KCl, 3 mM MgCl, 10 mM dithiothreitol, and 1.25 mM dNTP (dATP+dCTP+dGTP+dTTP), mixed with 200 units of a reverse transferase (GIBCO-BRL), and the resulting solution at a total volume of 20 μl was allowed to react at 42° C. for one hour. After the reaction solution underwent the phenol extraction followed by the ethanol precipitation, the thus-obtained pellets were dissolved in a mixed solution of 50 mM Tris-hydrochloric acid buffer solution (pH 7.5), 100 mM NaCl, 10 mM MgCl₂, and 1 mM dithiothreitol. Thereto were added 100 units of EcoRI and the resulting solution at a total volume of 20 μl was allowed to react at 37° C. for one hour. After the reaction solution underwent the phenol extraction followed by the ethanol precipitation, the obtained pellets were dissolved in a mixed solution of 20 mM Tris-hydrochloric acid buffer solution (pH 7.5), 100 mM KCl, 4 mM MgCl₂, 10 mM (NH₄)₂SO₄, and 50 μg/ml bovine serum albumin. Thereto were added 60 units of Escherichia coli DNA ligase and the resulting solution was allowed to react at 16° C. for 16 hours. To the reaction solution were added 2 μl of 2 mM dNTP, 4 units of Escherichia coli DNA polymerase I, and 0.1 unit of Escherichia coli DNase H and the resulting solution was allowed to react at 12° C. for one hour and then at 22° C. for one hour.

Next, the cDNA-synthesis reaction solution was used to transform Escherichia coli DH12S (GIBCO-BRL). The transformation was carried out by the electroporation method. A portion of the transformant was inoculated on a 2×YT agar culture medium containing 100 μg/ml ampicillin, which was incubated at 37° C. overnight. A colony grown on the culture medium was randomly picked up and inoculated on 2 ml of the 2×YT culture medium containing 100 μg/ml ampicillin, which was incubated at 37° C. overnight. The culture medium was centrifuged to separate the cells, from which a plasmid DNA was prepared by the alkaline lysis method. After the plasmid DNA was double-digested with EcoRI and NotI, the product was subjected to 0.8% agarose gel electrophoresis to determine the size of the cDNA insert. In addition, by the use of the obtained plasmid as a template, the sequence reaction using M13 universal primer labeled with a fluorescent dye and Taq polymerase (a kit of Applied Biosystems Inc.) was carried out and the product was analyzed by a fluorescent DNA-sequencer (Applied Biosystems Inc.) to determine the base sequence of the cDNA 5′-terminal of about 400 bp. The sequence data were filed as a homo-protein cDNA bank data base.

(3) Selection of cDNAs Encoding Proteins Having Transmembrane Domains

The base sequence registered in the homo-protein cDNA bank was converted to three frames of amino acid sequences and the presence or absence of an open reading frame (ORF) beginning from the initiation codon. Then, the selection was made for the presence of a signal sequence that is characteristic to a secretory protein at the N-terminal of the portion encoded by ORF. These clones were sequenced from the both 5′ and 3′ directions by using the deletion method to determine the whole base sequence. The hydrophobicity/hydrophilicity profiles were obtained for proteins encoded by ORF by the Kyte-Doolittle method [Kyte, J. & Doolittle, R. F., J. Mol. Bio. 157: 105-132 (1982)] to examine the presence or absence of a hydrophobic region. In the case in which there is a hydrophobic region of putative transmembrane domain(s) in the amino acid sequence of an encoded protein, this protein was considered as a membrane protein.

(4) Construction of Secretory Signal Detection Vector pSSD3

One microgram of pSSD1 carrying the SV40 promoter and a cDNA encoding the protease domain of urokinase [Yokoyama-Kobayashi, M. et al., Gene 163: 193-196 (1995)] was digested with 5 units of Bg1II and 5 units of EcoRV. Then, after dephosphorylation at the 5′ terminal by the CIP treatment, a DNA fragment of about 4.2 kbp was purified by cutting off from the gel of agarose gel electrophoresis.

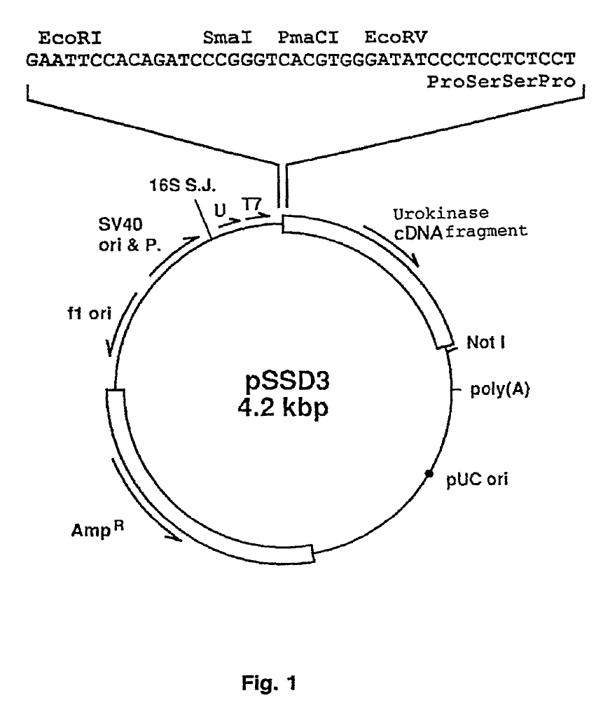

Two oligo DNA linkers, L1 (5′-GATCCCGGGTCACGTGGGAT-3′) and L2 (5′-ATCCCACGTGACCCGG-3′), were synthesized and phosphorylated by T4 polynucleotide kinase. After annealing of the both linkers, followed by ligation with the previously-prepared pSSD1 fragment by T4 DNA ligase, Escherichia coli JM109 was transformed. A plasmid pSSD3 was prepared from the transformant and the objective recombinant was confirmed by the determination of the base sequence of the linker-inserted fragment. FIG. 1 illustrates the structure of the thus-obtained plasmid. The present plasmid vector carries three types of blunt-end formation restriction enzyme sites, SmaI, PmaCI, and EcoRV. Since these cleavage sites are positioned in succession at an interval of 7 bp, selection of an appropriate site in combination of three types of frames for the inserting cDNA allows to construct a vector expressing a fusion protein.

(5) Functional Verification of Secretory Signal Sequence

Whether the N-terminal hydrophobic region in the secretory protein clone candidate obtained in the above-mentioned steps functions as the secretory signal sequence was verified by the method described in the literature [Yokoyama-Kobayashi, M. et al., Gene 163: 193-196 (1995)]. First, the plasmid containing the target cDNA was cleaved at an appropriate restriction enzyme site that existed at the downstream of the portion expected for encoding the secretory signal sequence. In the case in which this restriction enzyme site was a protruding terminus, the site was blunt-ended by the Klenow treatment or treatment with the mung-bean nuclease. Digestion with HindIII was further carried out and a DNA fragment containing the SV40 promoter and a cDNA encoding the secretory sequence at the downstream of the promoter was separated by agarose gel electrophoresis. This fragment was inserted between the pSSD3 HindIII site and a restriction enzyme site selected so as to match with the urokinase-coding frame, thereby constructing a vector expressing a fusion protein of the secretory signal portion of the target cDNA and the urokinase protease domain.

After Escherichia coli (host: JM109) bearing the fusion-protein expression vector was incubated at 37° C. for 2 hours in 2 ml of the 2×YT culture medium containing 100 μg/ml ampicillin, the helper phage M13KO7 (50 μl) was added and the incubation was continued at 37° C. overnight. A supernatant separated by centrifugation underwent precipitation with polyethylene glycol to obtain single-stranded phage particles. These particles were suspended in 100 μl of 1 mM Tris-0.1 mM EDTA, pH 8 (TE). Also, there was used as a control a suspension of single-stranded particles prepared in the same manner from the vector pKA1-UPA containing pSSD3 and a full-length cDNA of urokinase [Yokoyama-Kobayashi, M. et al., Gene 163: 193-196 (1995)].

The simian-kidney-origin culture cells, COS7, were incubated at 37° C. in the presence of 5% CO₂ in the Dulbecco's modified Eagle's culture medium (DMEM) containing 10% fetal calf albumin. Into a 6-well plate (Nunc Inc., 3 cm in the well diameter) were inoculated 1×10⁵ COS7 cells and incubation was carried out at 37° C. for 22 hours in the presence of 5% CO₂. After the culture medium was removed, the cell surface was washed with a phosphate buffer solution and then washed again with DMEM containing 50 mM Tris-hydrochloric acid (pH 7.5) (TDMEM). To the cells were added 1 μl of the single-stranded phage suspension, 0.6 ml of the DMEM culture medium, and 3 μl of TRANSFECTAM™ (IBF Inc.) and the resulting mixture was incubated at 37° C. for 3 hours in the presence of 5% CO₂. After the sample solution was removed, the cell surface was washed with TDMEM, 2 ml per well of DMEM containing 10% fetal calf albumin was added, and the incubation was carried out at 37° C. for 2 days in the presence of 5% CO₂.

To 10 ml of 50 mM phosphate buffer solution (pH 7.4) containing 2% bovine fibrinogen (Miles Inc.), 0.5% agarose, and 1 mM potassium chloride were added 10 units of human thrombin (Mochida Pharmaceutical Co., Ltd.) and the resulting mixture was solidified in a plate of 9 cm in diameter to prepare a fibrin plate. Ten microliters of the culture supernatant of the transfected COS7 cells were spotted on the fibrin plate, which was incubated at 37° C. for 15 hours. The diameter of the thus-obtained clear circle was taken as an index for the urokinase activity. In the case in which a cDNA fragment codes for the amino acid sequence that functions as a secretory signal sequence, a fusion protein is secreted to form a clear circle by its urokinase activity. Therefore, in the case in which a clear circle is not formed, the fusion protein remains as trapped in the membrane and the cDNA fragment is considered to code for a transmembrane domain.

(6) Protein Synthesis by In Vitro Translation

The plasmid vector carrying the cDNA of the present invention was utilized for the in vitro transcription/translation by the T_{N}T rabbit reticulocyte lysate kit (Promega Biotec). In this case, [³⁵S]methionine was added and the expression product was labeled with the radioisotope. All reactions were carried out by following the protocols attached to the kit. Two micrograms of the plasmid was allowed to react at 30° C. for 90 minutes in total 25 ml of a reaction solution containing 12.5 μl of the T_{N}T rabbit reticulocyte lysate, 0.5 μl of the buffer solution (attached to the kit), 2 μl of an amino acid mixture (methionine-free), 2 μl (0.37 MBq/μl) of [³⁵ S]methionine (Amersham Corporation), 0.5 μl of T7 RNA polymerase, and 20 U of RNasin. To 3 μl of the reaction solution was added 2 μl of an SDS sampling buffer (125 mM Tris-hydrochloric acid buffer solution, pH 6.8, 120 mM 2-mercaptoethanol, 2% SDS solution, 0.025% bromophenol blue, and 20% glycerol) and the resulting solution was heated at 95° C. for 3 minutes and then subjected to SDS-polyacrylamide gel electrophoresis. The molecular weight of the translation product was determined by carrying out the autoradiography.

(7) Northern Blot Hybridization

The northern blot hybridization was carried out in order to examine the expression pattern in the human tissues. Membranes on which poly(A)⁺ RNAs isolated from each of the human tissues are blotted are purchased from Clontech Inc. cDNA fragments which were excised from the objective clones with appropriate restriction enzymes were subjected to separation by agarose gel electrophoresis followed by labeling with [³²P] dCPT (Amersham Corporation) using the Random Primer Labeling Kit (Takara Shuzo Co., Ltd.). Hybridization was carried out using a solution attached to the blotted membrane in accordance to the protocol.

(8) Expression in COS7

Escherichia coli having an expression vector of the protein of the invention was infected with helper phage M13KO7, and single stranded phage was obtained by the above method. Using the thus obtained phage, the expression vector was introduced into simian kidney-originated culture cells COS7 according to the above method. Cultivation was carried out at 37° C. in the presence of 5 % CO₂ for 2 hours and then in a medium containing [³⁵S]cysteine for 1 hour. The cells were collected, dissolved and subjected to SDS-PAGE, whereby a band corresponding to a protein as the expression product, which was not present in the COS cells, was revealed.

(9) Clone Examples

<HP00442> (Sequence Number 1, 26, 51)

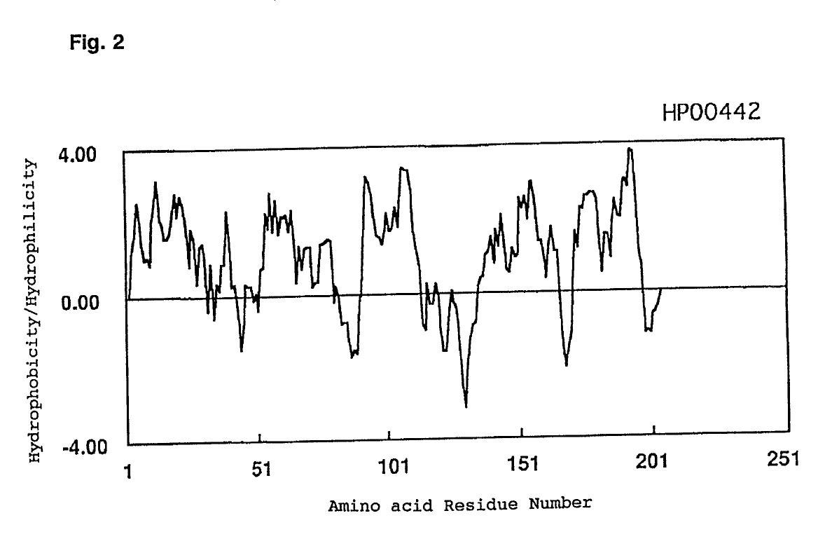

Determination of the whole base sequence for the cDNA insert of clone HP00442 obtained from the human fibrosarcoma cell line HT-1080 cDNA libraries revealed the structure consisting of a 5′-non-translation region of 81 bp, an ORF of 618 bp, and a 3′-non-translation region of 287 bp. The ORF codes for a protein consisting of 205 amino acid residues with 5 transmembrane domains. FIG. 2 depicts the hydrophobicity/hydrophilicity profile of the present protein obtained by the Kyte-Doolittle method. The result of the in vitro translation did not reveal the formation of distinct bands for the translation products and revealed the formation of smeary bands at the high-molecular-weight position.

The search of the protein data base using the amino acid sequence of the present protein revealed that the protein was analogous to the proteolipid protein PPA1 of the baker's yeast proton ATPase (SWISS-PROT Accession No. P23968). Table 2 indicates the comparison of the amino acid sequences between the human protein of the present invention (HP) and the proteolipid protein PPA1 of the baker's yeast proton ATPase (PL). - represents a gap, * represents an amino acid residue identical to that in the protein of the present invention, and . represents an amino acid residue analogous to that in the protein of the present invention. The both proteins possessed a homology of 56.8% in the entire region except for the N-terminal.

TABLE 2

| | |
|---|---|
| HP | MTGLALLYSGVFVAFWACALAVGVCYTIF-DLGFRFDVAWFLTETSPFMWS |
| | *..* . * .. .** ***.**. |
| PL | MNKESKDDDMSLGKFSFSHFLYYLVLTVVIVYGLYKLFTGHGSDINFGKFLLRTSPYMWA |
| HP | NLQIGLAISLSVVGAAWGIYITGSSIIGGGVKAPRIKTKNLVSIIFCEAVAIYGIIMAIV |
| | ****.* ..**********.*****.**.**.****.****.******.*****.*.*** |
| PL | NLGIALCVGLSVVGAAWGIFITGSSMIGAGVRAPRITTKNLISIIFCEVVAIYGLIIAIV |
| HP | ISNMAEPFSATDPKAIGHRNYHAGYSMFGAGLTVGLSNLFCGVCVGIVGSGAALADAQNP |
| | .*.. **. . ...* ..***.*.**.***.***.**.***.*..**..**... |
| PL | FSSKL--TVATAENMYSKSNLYTGYSLFWAGITVGASNLICGIAVGITGATAAISDAADS |
| HP | SLFVKILIVEIFGSAIGLFQVIVAILQTSRVKMGD |
| | .******..***** .**.*.**..* ... |
| PL | ALFVKILVIEIFGSILGLLGLIVGLLMAGKASEFQ |

Furthermore, the search of GenBank using the base sequence of the present cDNA revealed that there existed some ESTs possessing the homology of 90% or more and also containing the initiation codon (for example, Accession No. H87379), but the present protein can not be predicted from this sequence.

The proteolipid protein PPA1 of the baker's yeast proton ATPase is a membrane protein essential to the growth of cells [Apperson, M. et al., Biochem. Biophys. Res. Commun. 168: 574-579 (1990)]. Accordingly, the protein of present invention, which is homologous to said protein, is considered to be essential to the growth of human cells and can be utilized for the diagnosis and the treatment of diseases caused by the abnormality of the present protein.

<HP00804> (Sequence Number 2, 27, 52)

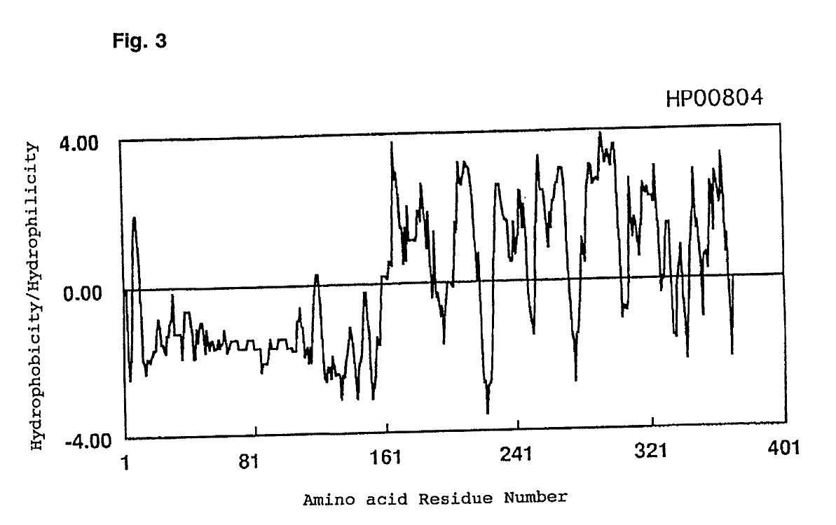

Determination of the whole base sequence for the cDNA insert of clone HP00804 obtained from the human leukocyte cell cDNA libraries revealed the structure consisting of a 5′-non-translation region of 132 bp, an ORF of 1116 bp, and a 3′-non-translation region of 576 bp. The ORF codes for a protein consisting of 371 amino acid residues with 7 transmembrane domains. FIG. 3 depicts the hydrophobicity/hydrophilicity profile of the present protein obtained by the Kyte-Doolittle. The result of the in vitro translation did not reveal the formation of distinct bands for the translation products.

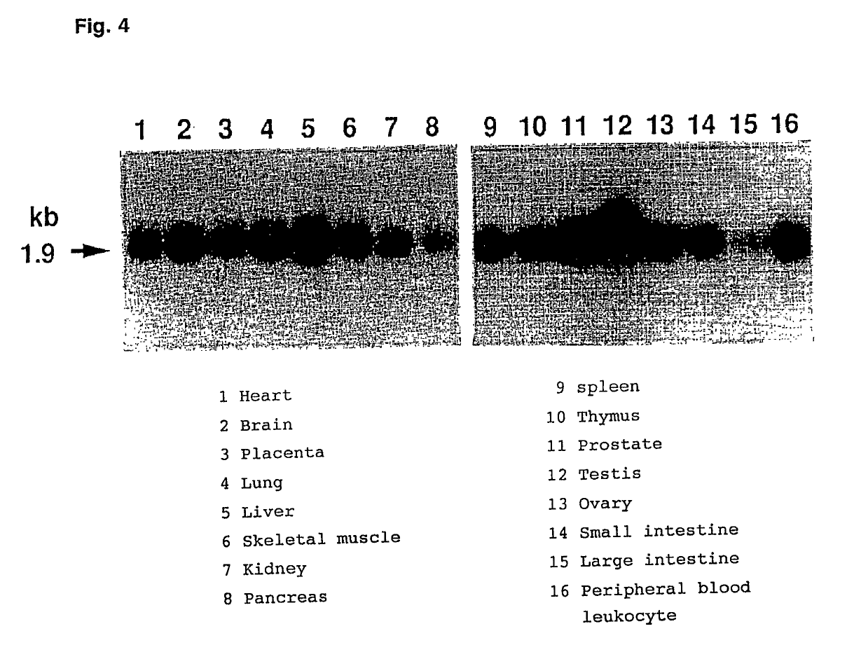

Examination of the expression pattern in the tissues by the northern blot hybridization using the cDNA fragment of the present invention revealed that the expression occurred in all tissues examined as shown in FIG. 4. Therefore, the protein of the present invention is considered to be a housekeeping protein.

The search of the protein data base using the amino acid sequence of the present protein revealed that the protein was analogous to the rat NMDA receptor - glutamate-binding subunit (GenBank Accession No. S61973). Table 3 indicates the comparison of the amino acid sequences between the human protein of the present invention (HP) and the rat NMDA receptor - glutamate-binding subunit (RN). - represents a gap, * represents an amino acid residue identical to that in the protein of the present invention, and represents an amino acid residue analogous to that in the protein of the present invention. This subunit consists of 516 amino acid residues and a region from glutamine at position 68 to arginine at position 342 possessed a 92.6% homology with the C-terminal 270 amino acid residues in the protein of the present invention. However, any homology was not observed in the N-terminal region. Hereupon, a characteristic repeated sequence that is rich with proline, tyrosine, and glycine was observed in the N-terminal region of the protein of the present invention.

TABLE 3

| | |
|---|---|
| HP | MSHEKSFLVSGDNYPPPNPGYPGGPQPPMPPYAQPPYPGAPYPQPPFQPSPYGQPQYPHG |
| RN | MKRVSWSLGTAILPQTLAILWGHKPLCLPMFSLPTLG |
| HP | PSPYPQGGYFQGPYPQGGYPQGPYPQEGYPQGPYPQGGYPQGPYPQSPFPPNPYGQPQVF |
| | **.**************. * |
| RN | PHTHRPLSSPLPMVNQGIPMVPVPITRWLPLKDLLKEATHQGHYPQSPFPPNPYGQPPPF |
| HP | PGQDPDSPQHCNYQEEGPPSYYDNQDFPATNWDDKSIRQAFIRKVFLVLTLQLSVTLSTV |
| | ***.**********************.. *************************** |
| RN | --QDPGSPQHGNYQEEGPPSYYDNQDFPSVNW-DKSIRQAFIRKVFLVLTLQLSVTLSTV |
| HP | SVFTFVAEVKGFVRENVWTYYVSYAVFFISLIVLSCCGDFRRKHPWNLVALSVLTASLSY |
| | ..****.*******.**********.**************.**********.** *** |
| RN | AIFTFVGEVKGFVRANVWTYYVSYAIFFISLIVLSCCGDFRKKHPWNLVALSILTISLSY |
| HP | MVGMIASFYNTEAVIMAVGITTAVCFTVVIFSMQTRYDFTSCMGVLLVSMVVLFIFAILC |
| | ************************************************.********** |
| RN | MVGMIASFYNTEAVIMAVGITTAVCFTVVIFSMQTRYDFTSCMGVLLVSVVVLFIFAILC |
| HP | IFIRNRILEIVYASLGALLFTCFLAVDTQLLLQNKQLSLSPEEYVFAALNLYTDIINIFL |
| | ********************************************************** |
| RN | IFIRLNRILEIVYASLQALLFTCFLAVDTQLLLGNKQLSLSPEEYVFAALNLYTDIINIFL |
| HP | YILTIIGRAKE |
| RN | YILTIIGRSQGIGQAPAQVAWAQTHAPAMTLPSVLPPLWFPAMAWSRGSPSRPRVCTLQ |

Furthermore, the search of GenBank using the base sequence of the present cDNA revealed that there existed some ESTs possessing the homology of 90% or more (for example, Accession No. W25936), but any of them was shorter than the present cDNA and did not contain the initiation codon.

The rat NMDA receptor - glutamate-binding subunit has been found as one of the subunits of the NMDA receptor complex which exists specifically in the brain [Kumar. K. N. et al., Nature 354: 70-73 (1991)]. Despite a high homology with the protein of the present invention, the subunit shows different expression patterns in the N-terminal sequence and the tissues, whereby both molecules are considered to possess different functions. Since the protein of the present invention possesses 7 transmembrane domains which are characteristic to channels and transporters, this protein is considered to play a role as a channel and a transporter. Because the protein of the present invention is a housekeeping protein essential to the cells, the present protein can be utilized for the diagnosis and the treatment of diseases caused by the abnormality of this protein.

<HP01098> (Sequence Number 3, 28, 53)

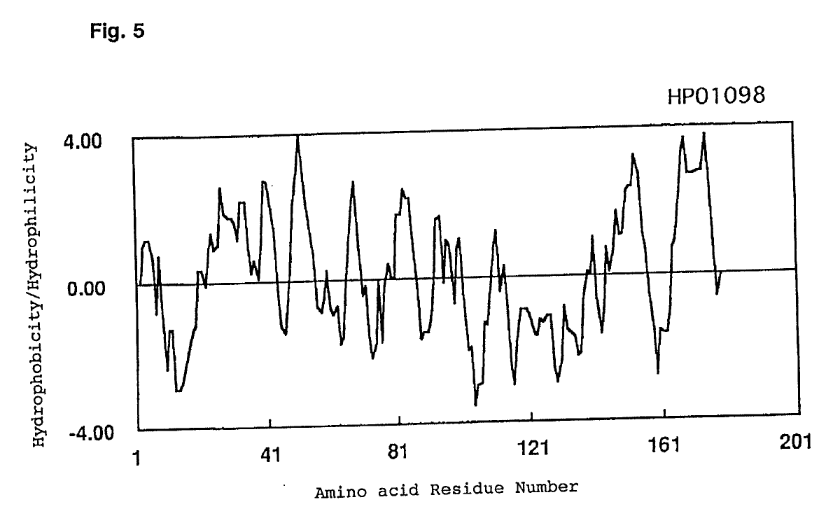

Determination of the whole base sequence for the cDNA insert of clone HP01098 obtained from the human stomach cancer cDNA libraries revealed the structure consisting of a 5′-non-translation region of 61 bp, an ORF of 540 bp, and a 3′-non-translation region of 475 bp. The ORF codes for a protein consisting of 179 amino acid residues with one transmembrane domain. FIG. 5 depicts the hydrophobicity/hydrophilicity profile of the present protein obtained by the Kyte-Doolittle method. The in vitro translation resulted in the formation of a translation product of 20 kDa that was almost consistent with the molecular weight of 20,625 predicted from the ORF.

The search of the protein data base using the amino acid sequence of the present protein revealed that the protein was completely identical with a 18-kDa subunit of the canine microsomal signal peptidase (SWISS-PROT Accession No. P21378). Therefore, it was verified that the cDNA of the present invention codes for the human homologue of the 18-kDa subunit of the microsomal signal peptidase.

The search of GenBank using the base sequence of the present cDNA revealed that there existed some ESTs possessing the homology of 90% or more (for example, Accession No. T60549), but many sequences were not distinct and the same ORF as that in the present cDNA was not identified.

The 18-kDa subunit of the canine microsomal signal peptidase has been found as one of subunits of the signal peptidase complex that exist in the microsome [Schelness, G. S. & Blobel, G., J. Biol. Chem. 265: 9512-9519 (1990)]. The signal peptidase is an enzyme that cleaves the signal sequence upon secretion of a secretory protein at the endoplasmic reticulum. Therefore, the cDNA of the present invention can be utilized for the production of the present protein as well as for the diagnosis and the treatment of diseases caused by the abnormality of the present protein.

<HP01148> (Sequence Number 4, 29, 54)

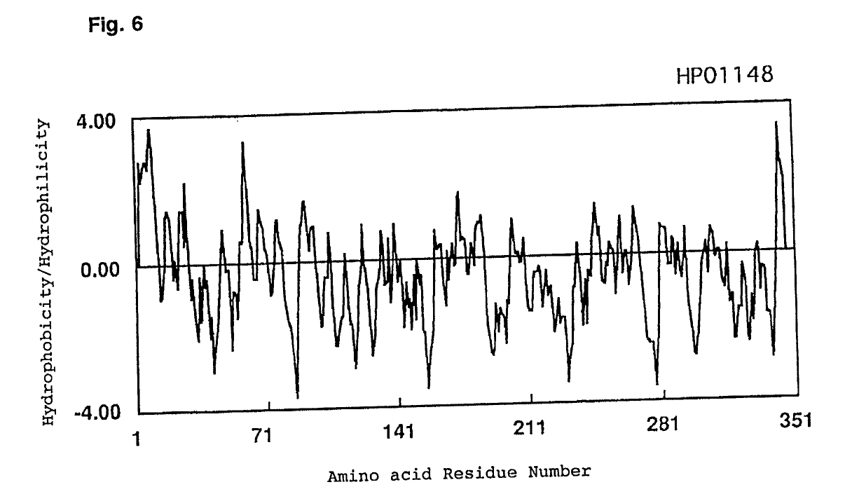

Determination of the whole base sequence for the cDNA insert of clone HP01148 obtained from the human liver cDNA libraries revealed the structure consisting of a 5′-non-translation region of 101 bp, an ORF of 1044 bp, and a 3′-non-translation region of 446 bp. The ORF codes for a protein consisting of 347 amino acid residues with one transmembrane domain at the N-terminal. FIG. 6 depicts the hydrophobicity/hydrophilicity profile of the present protein obtained by the Kyte-Doolittle method. It was indicated that the present protein remained in the membrane from the observation that the urokinase secretion was not identified, upon transduction into the COS7 cells of an expression vector in which a HindIII-PvuII fragment containing a cDNA fragment encoding the N-terminal 178 amino acid residues in the present protein was inserted at the HindIII-PmaCI site of pSSD3. Therefore, the present protein is considered to be a type-II membrane protein. The in vitro translation resulted in the formation of a translation product of 41 kDa that was almost consistent with the molecular weight of 38,101 predicted from the ORF.

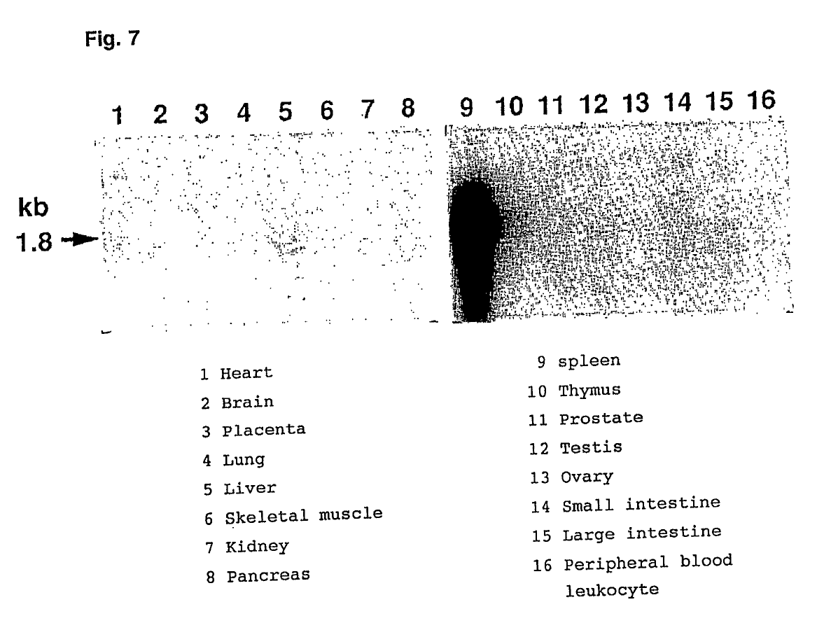

Examination of the expression pattern in the tissues by the northern blot hybridization using the cDNA fragment of the present invention revealed that a strong expression occurred in the spleen, as shown in FIG. 7. It was also indicated that a slight expression occurred in the liver.

The search of the protein data base using the amino acid sequence of the present protein revealed that the protein was analogous to the bovine WC1 antigen (SWISS-PROT Accession No. P30205). Table 4 indicates the comparison of the amino acid sequences between the human protein of the present invention (HP) and the bovine WC1 antigen (WC). - represents a gap, * represents an amino acid residue identical to that in the protein of the present invention, and . represents an amino acid residue analogous to that in the protein of the present invention. The both proteins possessed a homology of 38%.

TABLE 4

| | | | | |
|---|---|---|---|---|
| HP | MALLFSLILAICTRPGFLASPSGVRLVGGLHRCEGRVEVEQKGQWQTVCDDGW | | | |
| | . .... .***.* .*.****. ..* ***.***.* | | | |
| WC | VLPQCNDFLSQPAQSAASEESSPYCSDSRQLRLVDGGQPCGGRVEILDQGSWGTICDDDW | | | |
| HP | DIKDVAVLCRELGCGAASGTPSGILYEPPAEKEQKVLIQSVSCTGTEDTLAQCEQEE--V | | | |
| | *..*. *.**.***.* ..... . .. . . .....***.*. . .* . | ***.*. | | |
| WC | DLDDARVVCRQLGCGEALNATGSAHF---GAGSGPTWLDDLNCTGKESHVWRCPSRGWGR | | | |
| HP | YDCSHEEDAGASCENPESSFSPVPEGVRLADGPGHCKQRVEVKHQNQWYTVCQTGWSLRA | | | |
| | .**.*.****. * .* * ..*.... .* *..** ... * .**.. . . | * .* * | . .* | ** ... * .**.. |
| WC | HDCREKRDAGVIC--SE--F----LALRMVSEDQQCAGWLEVFYNGTWGSVCRSPMEDIT | | | |
| HP | AKVVCRQLGCGRAVLTQKRCNHAYGRKPIWLSQMSCSGREATLQDCPSGPWGKNTCNHD | | | |
| | ..*.******* . ... . .. *..* *.. . *....* .****** ..*... | * .****** . | | |
| WC | VSVICRQLGCGDSGSLNTSVGLRE-GSRPRWVDLIQGRKHDTSLWQCPSGPWKYSSCSPK | | | |
| HP | EDTWVECE-------------DPFDLRLVGGDNLCSGRLEVLHKGVWGSVCDDNWGEKE | | | |
| | *.....** *. .*** ***.****.** *.* **.****.*. * | *. .*** ***. ****.** *.* **.****.*. * | | |
| WC | EEAYISCEQRRPKSCPTAAACTDREKLRLRGGDSECSGRVEVWHNQSWGTVCDDSWSLAE | | | |
| HP | DQVVCKQLQCGKSLSPSFRDRKCYGPCVGRTWLDNVRCSGEEQSLEQCQHRFWGFHDCTH | | | |
| | ..***.*****..*. . * . .*** *.****.*.*.* * ** .* ** **.* | .*** *.****.*.*.* * ** .* | ** **.* | |
| WC | AEVVCQQLGCGQALE-AVR-SAAFGPGNGSIWLDEVQCGGRESSLWDCVAEPWCQSDCKH | | | |
| HP | QEDVAVICSG | | | |
| | .**..* *** | | | |
| WC | EEDAGVRCSGVRTTLPTTTAGTRTTSNSLPGIFSLPGVLCLILGSLLFLVLVILVTQLLR | | | |

Furthermore, the search of GenBank using the base sequence of the present cDNA revealed that there existed some ESTs possessing the homology of 90% or more (for example, Accession No. H91200), but it can not be assessed whether these ESTs with partial sequences code for the same protein as the protein of the present invention.

The bovine WC1 antigen has been found as a membrane antigen which is expressed specifically in γδ T cells [Wijngaard, P. L. J. et al., J. Immunol. 149: 3273-3277 (1992)]. The region showing an analogy is called the scavenger receptor cysteine-rich domain (SRCR) which also exists as a repeated sequence in macrophage scavenger receptors [Matsumoto, A. et al., Proc. Natl. Acad. Sci. USA 87: 9133-9137 (1990)], T cell differentiation antigen CD6 [Aruffo, A. et al., J. Exp. Med. 174: 949-952 (1991)], and so on. Since the present protein is expressed specifically in the spleen, This protein is considered to be deeply associated with the functions of the spleen and also to function as a receptor in the same manner as other SRCR family members.

<HP01293> (Sequence Number 5, 30, 55)

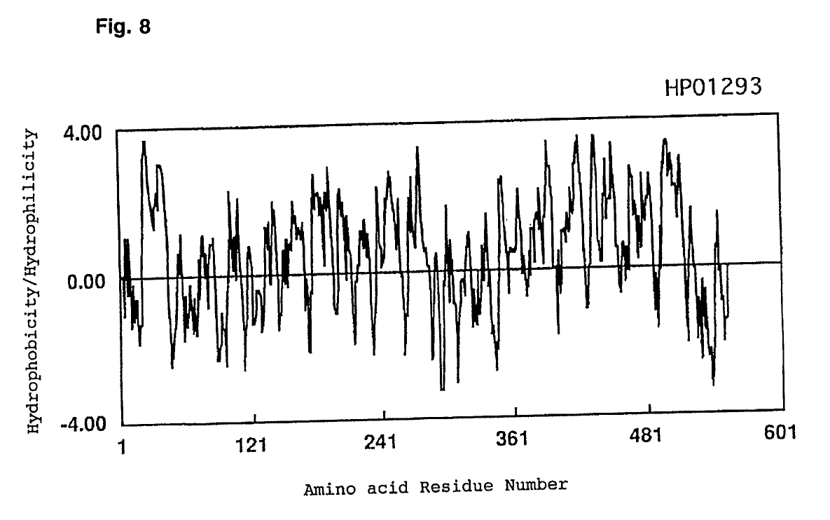

Determination of the whole base sequence for the cDNA insert of clone HP01293 obtained from the human liver cDNA libraries revealed the structure consisting of a 5′-non-translation region of 89 bp, an ORF of 1665 bp, and a 3′-non-translation region of 134 bp. The ORF codes for a protein consisting of 554 amino acid residues with 12 transmembrane domains. FIG. 8 depicts the hydrophobicity/hydrophilicity profile of the present protein obtained by the Kyte-Doolittle method. The in vitro translation did not reveal the formation of distinct bands and revealed the formation of smeary bands at the high-molecular-weight position.

The search of the protein data base using the amino acid sequence of the present protein revealed that the protein was analogous to the rat cation transporter (GenBank Accession No. X78855). Table 5 indicates the comparison of the amino acid sequences between the human protein of the present invention (HP) and the mouse interstitial cell protein (MM). - represents a gap, * represents an amino acid residue identical to that in the protein of the present invention, and . represents an amino acid residue analogous to that in the protein of the present invention. The both proteins possessed a homology of 78.1% among the entire regions.

TABLE 5

| | |
|---|---|
| HP | MPTVDDILEQVGESGWFQKQAFLILCLLSAAFAPICVGIVFLGFTPDBBCQSPGVAELSQ |
| | ******.****** *********.***.**..***.**********.*.**.******** |
| RN | MPTVDDVLEQVGEFGFQKQAFLLLCLISASLAPIYVGIVFLGFTPGHYCQNPGVAELSQ |
| HP | RCGWSPAEELNYTVPGLGPAGEA-FLGQCRRYEVDWNQSALSCVDPLASLATNRSHLPLG |
| | *****.*************..** **.**.*********.*.*****.**..***.**** |
| RN | RCGWSQAEELNTVPGLGPSDEASFLSQCMRYEVDWNQSTLDCVDPLSSLVANRSQLPLG |
| HP | PCQDGWVYDTPGSSIVTEFNLVCADSWKLDLFQSCLNAGFFFGSLGVGYFADRFGRKLCL |
| | **..*******************.*.**.******.* ***.*** ***.********** |
| RN | PCEHGWVYDTPGSSIVTEFNLVCGDAWKVDLFQSCVNLGFFLGSLVVGYIADRFGRKLCL |
| HP | LGTVLVNAVSGVLMAFSPNYMSMLLFRLLQGLVSKGNWMAGYTLITEFVGSGSRRTVAIM |
| | * *.**..***** * .*.* **********.****.*..********** ***.**. |
| RN | LVTTLVTSVSGVLTAVAPDYTSMLLFRLLQGMTSKGSWVSGYTLITEFVGSGYRRTTAIL |
| HP | YQMAFTVGLVALTGLAYALPEWRWLQLAVSLPTFLFLLYYWCVPESPRWLLSQKRNTEAI |
| | **********.*.*.***.*.******************** *************.* *. |
| RN | YQMAFTVGLVGLAGVAYAIPDWRWLQLAVSLPTFLFLLYYWFVPESPRWLLSQKRTTRAV |
| HP | KIMDHIAQKNGKLPPADLKMLSLEEDVTEKLSPSFADLFRTPRLRKRTFILMYIWFTDSV |
| | .**..*******.********.****..** ***********.***.* *******. .* |
| RN | RIMEQIAQKNGKVPPADLKMLCLEEDASEKRSPSFADLFRTPNLRKHTVILMYLWFSCAV |
| HP | LYQGLILHMGATSGNLYLDFLYSALVEIPGAFIALITIDRVGRIYPMAVSNLLAGAACLV |
| | ******.*.***..******.**.***.*.***.*.****.*****.*.***..*****. |
| RN | LYQGLIMHVGATGANLYLDFFYSSLVEFPAAFILVTIDRIGRIYPIAASNLVTGAACLL |
| HP | MIFISPDLHWLNIIIMCVGRMGITIAIQMICLVNAELYPTFVRNLGVMVCSSLCDIGGII |
| | ****...*****... *.**** **..**.***********.****.****.***.***. |
| RN | MIFIPHELHWLNVTLACLGRMGATIVLQMVCLVNAELYPTFIRNLGMMVCSALCDLGGIF |
| HP | TPFIVFRLREVWQALPLILFAVLGLLAAGVTLLLPETKGVALPETMKDEANLG-RKAKPK |
| | ***.****.***********.**** *...***************...***** **.*.* |
| RN | TPFMVFRLMEVWQALPLILFGVLGLTAGAMTLLLPETKGVALPETIEEAENLGRRKSKAK |
| HP | ENTIYLKVQTSEPSGT |
| | ******.***...*.* |
| RN | ENTIYLQVQTGKSSST |

Furthermore, the search of GenBank using the base sequence of the present cDNA revealed that there did not exist any human gene and human EST possessing the homology of 90% or more.

The rat cation transporter has been found as a membrane protein that relates to the drug excretion in the kidney [Grundemann, D. et al., Nature 372: 549-552 (1994)]. Accordingly, the protein of the present invention which is homologous to this transporter is considered to possess a similar function and can be utilized for the diagnosis and the treatment of diseases caused by the abnormality of this protein. In addition, since the present protein is considered to relate to the drug excretion, the cells in which this protein is expressed can be utilized as a tool for the drug design of these drugs. Furthermore, since the present protein is expressed principally in the liver and the kidney, a molecule that is prepared so as to possess an affinity to this protein is applicable for the drug delivery system into these tissues.

<HP10013> (Sequence Number 6, 31, 56)

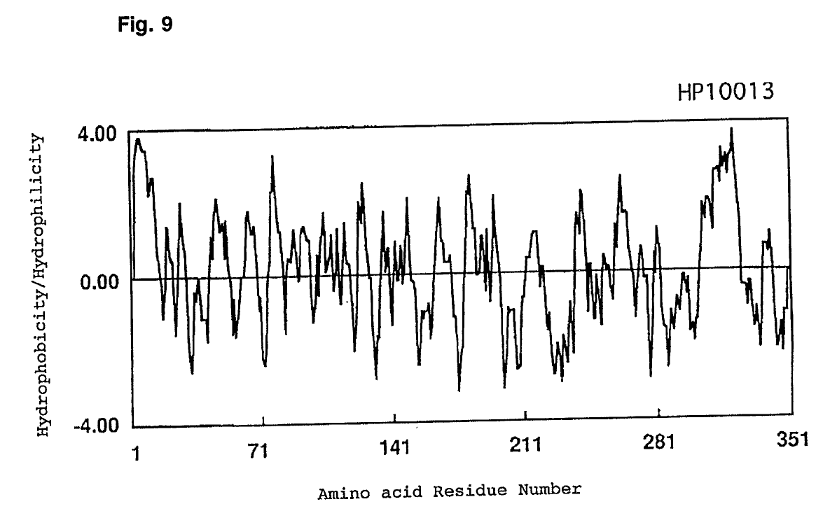

Determination of the whole base sequence for the cDNA insert of clone HP10013 obtained from the human epidermoid carcinoma cell line KB cDNA libraries revealed the structure consisting of a 5′-non-translation region of 96 bp, an ORF of 1053 bp, and a 3′-non-translation region of 884 bp. The ORF codes for a protein consisting of 350 amino acid residues with a signal sequence at the N-terminal and one internal transmembrane domain. FIG. 9 depicts the hydrophobicity/hydrophilicity profile of the present protein obtained by the Kyte-Doolittle method. It was indicated that the present protein functioned as a signal sequence at the N-terminal from the observation that the urokinase activity was detected in the culture medium, upon transduction into the COS7 cells of an expression vector in which a HindIII-EcoO65I fragment (treated with the mung-bean nuclease) containing a cDNA fragment encoding the N-terminal 65 amino acid residues in the present protein was inserted at the HindIII-EcoRV site of pSSD3. Therefore, the present protein is considered to be a type-I membrane protein. The in vitro translation resulted in the formation of a translation product of 39 kDa that was almost consistent with the molecular weight of 39,008 predicted from the ORF.

The search of the protein data base using the amino acid sequence of the present protein revealed that the protein was not analogous to any of known proteins. Furthermore, the search of GenBank using the base sequence of the present cDNA revealed that there existed some ESTs possessing the homology of 90% or more (for example, Accession No. H07998), but any of them was shorter than the present cDNA and did not contain the initiation codon.

<HP10034> (Sequence Number 7, 32, 57)

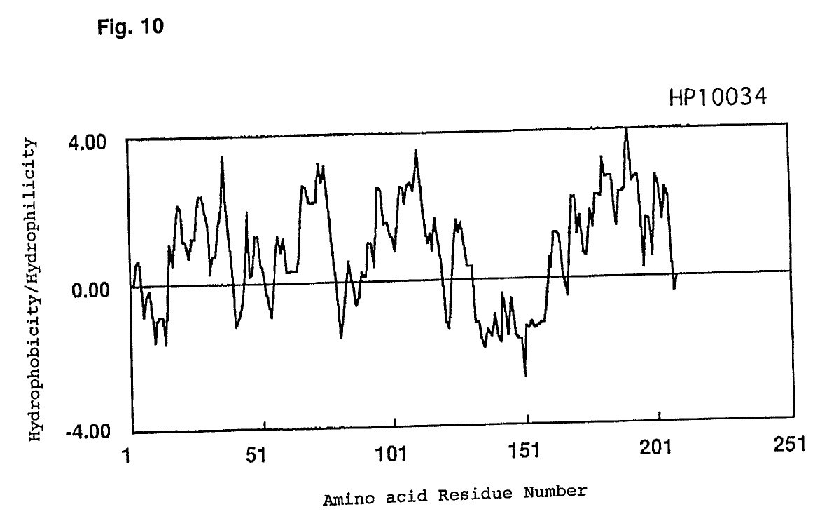

Determination of the whole base sequence for the cDNA insert of clone HP10034 obtained from the human fibrosarcoma cell line HT-1080 cDNA libraries revealed the structure consisting of a 5′-non-translation region of 175 bp, an ORF of 630 bp, and a 3′-non-translation region of 106 bp. The ORF codes for a protein consisting of 209 amino acid residues with 4 transmembrane domains. FIG. 10 depicts the hydrophobicity/hydrophilicity profile of the present protein obtained by the Kyte-Doolittle method. The in vitro translation resulted in the formation of a translation product of 21 kDa that was almost consistent with the molecular weight of 22,432 predicted from the ORF.

The search of the protein data base using the amino acid sequence of the present protein revealed that the protein was analogous to the human tumor-associated antigen L6 (SWISS-PROT Accession No. P30408). Table 6 indicates the comparison of the amino acid sequences between the human protein of the present invention (HP) and the human tumor-associated antigen L6 (L6). - represents a gap, * represents an amino acid residue identical to that in the protein of the present invention, and . represents an amino acid residue analogous to that in the protein of the present invention. The both proteins possessed a homology of 31.8%.

TABLE 6

| | | | |
|---|---|---|---|
| HP | MVSSPCTQASSRTCSRILGLSLQTAALFAAGANVALLLPNWDVTYLLRGLLQRHAMLGTG | | |
| | *.* .* ** . **. .**. * .** ...* .*.* . . .* | .**.* .** ...* .*.* . . | |
| L6 | MCYGKCARCIGHSLVGLALLCIAANILLYFPNGETKYASENHLSRFWAFFSG | | |
| HP | LWGGCLMVLTAA-ILISL-MGWRYGCFS--KSGLCRSVLTALLSGGLALLGALICFVTSG | | |
| | . ****..* .* ..*.* . .** . ..* ..*...*.. ... *. * | ..*...* | *. *.... |
| L6 | IVGGGLLNLLPAFVFIQLEQDDCCGCCGHENCGKRCAMLSSVLAALIGIAGSGYCVIVAA | | |
| HP | VALKDGPFCMFDVSSFNQTQAWKYGYPFKDLHSRNYLYDRSLWNSVCLEPSAAVVWHVSL | | |
| | ..* .**.*. * .*.*... .. .** * * *.. * **. * *.*** | | |
| L6 | LGLAEGPLCL-D-----SLGQWNYTFASTE---GQYLLDTSTWSE-CTEPKHIVEWNVSL | | |
| HP | FSALLCISLLQLLLVVVHVINSLLGLFCSLCEK | | |
| | ** ** .. ....* ...***..** .*..* | | |
| L6 | FSILLALGGIEFILCLIQVINGVLGGICGFCCSHQQQYDC | | |

Furthermore, the search of GenBank using the base sequence of the present cDNA revealed that there did not exist any human gene and human EST possessing the homology of 90% or more.

The human tumor-associated antigen L6 is a member of the membrane antigen TM4 super-family proteins that are expressed abundantly on the cell surface of human tumors [Marken, J. S. et al., Proc. Natl. Acad. Sci. USA 89: 3503-3507 (1992)]. Since these membrane antigens are expressed specifically in specific cells and in cancer cells, an antibody that is prepared so as to bind to this antigen is applicable for a variety of diagnoses and as a carrier for the drug delivery. Furthermore, cells in which such a membrane antigen is expressed by transduction of the membrane antigen gene are applicable to the detection of the corresponding ligand.

<HP10050> (Sequence Number 8, 33, 58)

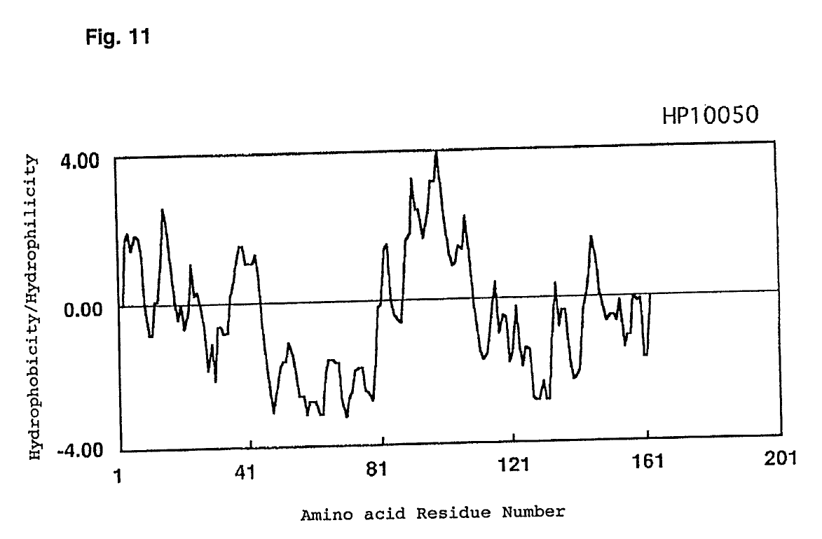

Determination of the whole base sequence for the cDNA insert of clone HP10050 obtained from the human fibrosarcoma cell line HT-1080 cDNA libraries revealed the structure consisting of a 5′-non-translation region of 9 bp, an ORF of 492 bp, and a 3′-non-translation region of 100 bp. The ORF codes for a protein consisting of 163 amino acid residues with one transmembrane domain. FIG. 11 depicts the hydrophobicity/hydrophilicity profile of the present protein obtained by the Kyte-Doolittle method. The in vitro translation resulted in the formation of a translation product of 23 kDa that was almost consistent with the molecular weight of 18,364 predicted from the ORF.

The search of the protein data base using the amino acid sequence of the present protein revealed that the protein was not analogous to any of known proteins. Furthermore, the search of GenBank using the base sequence of the present cDNA revealed that there existed some ESTs possessing the homology of 90% or more (for example, Accession No. H03117), but many sequences were not distinct and the same ORF as that in the present cDNA was not identified.

<HP10071> (Sequence Number 9, 34, 59)

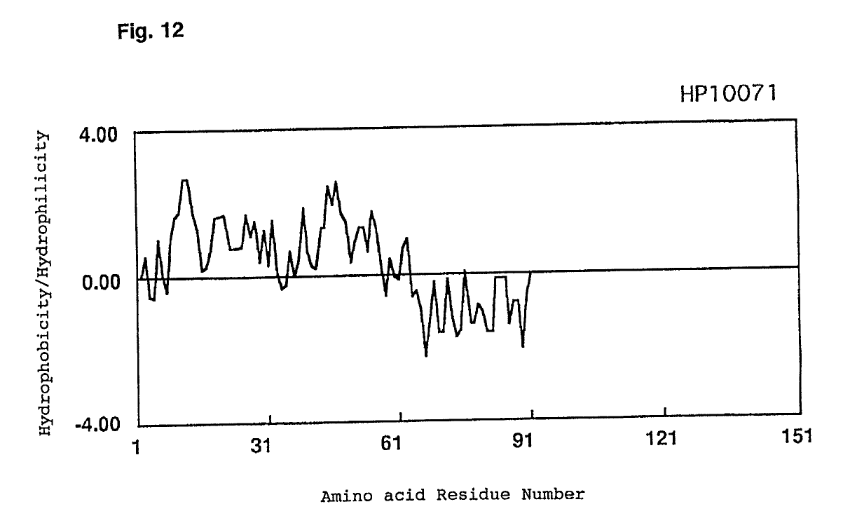

Determination of the whole base sequence for the cDNA insert of clone HP10071 obtained from the human stomach cancer cDNA libraries revealed the structure consisting of a 5′-non-translation region of 46 bp, an ORF of 279 bp, and a 3′-non-translation region of 69 bp. The ORF codes for a protein consisting of 92 amino acid residues with 2 transmembrane domains. FIG. 12 depicts the hydrophobicity/hydrophilicity profile of the present protein obtained by the Kyte-Doolittle method. The in vitro translation resulted in the formation of a translation product of 12 kDa that was almost consistent with the molecular weight of 10,094 predicted from the ORF.

The search of the protein data base using the amino acid sequence of the present protein revealed that the protein was not analogous to any of known proteins. Furthermore, the search of GenBank using the base sequence of the present cDNA revealed that there existed some ESTs possessing the homology of 90% or more (for example, Accession No. R097442), but many sequences were not distinct and the same ORF as that in the present cDNA was not identified.

<HP10076> (Sequence Number 10, 35, 60)

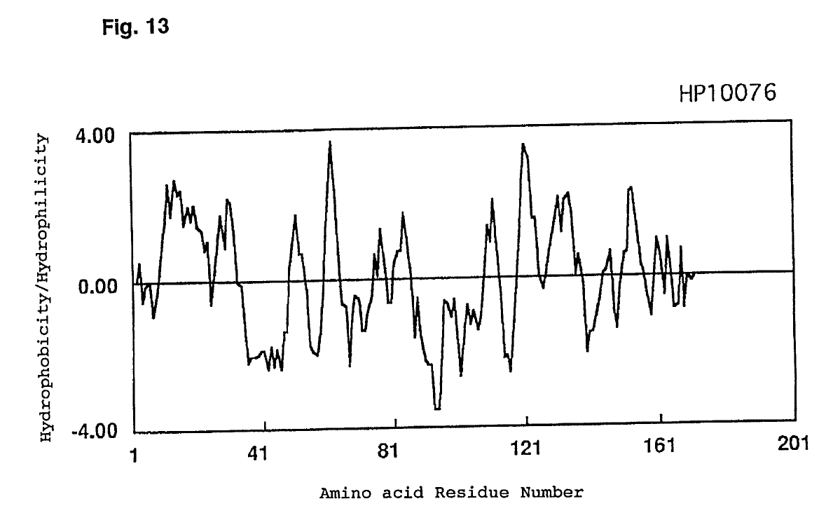

Determination of the whole base sequence for the cDNA insert of clone HP10076 obtained from the human lymphoma cell line U937 cDNA libraries revealed the structure consisting of a 5′-non-translation region of 81 bp, an ORF of 519 bp, and a 3′-non-translation region of 132 bp. The ORF codes for a protein consisting of 172 amino acid residues with 2 transmembrane domains. FIG. 13 depicts the hydrophobicity/hydrophilicity profile of the present protein obtained by the Kyte-Doolittle method. It was indicated that the present protein remained in the membrane from the observation that the urokinase secretion was not identified upon transduction into the COS7 cells of an expression vector in which a HindIII-EcoO651 (treated with mung-bean nuclease) fragment containing a cDNA fragment encoding the N-terminal 167 amino acid residues in the present protein was inserted at the HindIII-EcoRV site of pSSD3. The in vitro translation resulted in the formation of a translation product of 24 kDa that was almost consistent with the molecular weight of 18,450 predicted from the ORF.

The search of the protein data base using the amino acid sequence of the present protein revealed that the protein was analogous to the baker's yeast hypothetical membrane protein of 23.1 kDa (SWISS-PROT Accession No. P34222). Table 7 indicates the comparison of the amino acid sequences between the human protein of the present invention (HP) and the baker's yeast hypothetical membrane protein of 23.1 kDa (SC). - represents a gap, * represents an amino acid residue identical to that in the protein of the present invention, and . represents an amino acid residue analogous to that in the protein of the present invention. The both proteins possessed a homology of 47.5% in the C-terminal region of 139 amino acid residues.

TABLE 7

| | | |
|---|---|---|
| HP | MEYLAHPSTLGLAVGVACGMGLGWS | |
| SC | MITSFLMMTVSSNYTIALWATFTAISFAVGYQLGTSNASSTKKSSATLLRSKEMKEGK | |
| HP | LRVCFGMLPKSKTSKTHTDTESEASILGD-SGEYKMILVVRNDLKMGKGKVAAQCSHAAV | |
| | ...*.. *.. *.* .** .* **.*.** *.***.****.***. | |
| SC | LHNDTDEEESESEDESDEDEDIESTSLNDIPGEVRMALVIRQDLGMTKGKIAAQCCHAAL | |
| HP | SAYKQI-----QRRNPEMLKQWEYCGQPKVVVKAPDEETLIALLAHAKMLGLTVSLIQD | |
| | * ...* ..** * ..* **.*...* **. *. .* *.* **.....*.* | **.*...***.*. .**.* ** |
| SC | SCFRHIATNPARASYNPIMTQRWLNAGQAKITLKCPDKFTMDELYAKAISLGVNAAVIHD | |
| HP | AQRTQIAPCSQTVLGIGPGPADLIDKVTGHLKLY | |
| | *******.**.****.**.* ...*..**.**** | |
| SC | AGRTQIAAGSATVLQLCPAPKAVLDQITCDLKLY | |

Furthermore, the search of GenBank using the base sequence of the present cDNA revealed that there existed some ESTs possessing the homology of 90% or more (for example, Accession No. T74847), but many sequences were not distinct and the same ORF as that in the present cDNA was not identified.

<HP10085> (Sequence Number 11, 36, 61)

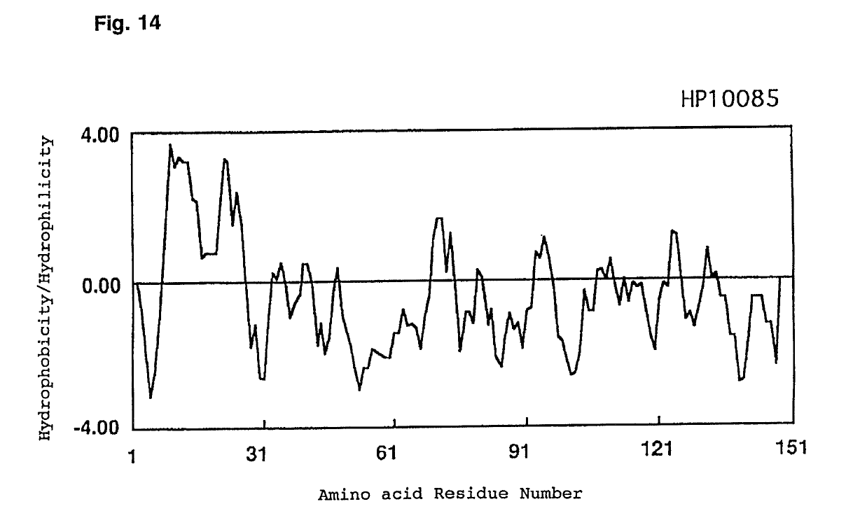

Determination of the whole base sequence for the cDNA insert of clone HP10085 obtained from the human lymphoma cell line U937 cDNA libraries revealed the structure consisting of a 5′-non-translation region of 150 bp, an ORF of 450 bp, and a 3′-non-translation region of 97 bp. The ORF codes for a protein consisting of 149 amino acid residues with one transmembrane domain at the N-terminal. FIG. 14 depicts the hydrophobicity/hydrophilicity profile of the present protein obtained by the Kyte-Doolittle method. It was indicated that the present protein remained in the membrane from the observation that the urokinase secretion was not identified upon transduction into the COS7 cells of an expression vector in which a HindIII-EcoRI fragment (after the Klenow treatment) containing a cDNA fragment encoding the N-terminal 57 amino acid residues in the present protein was inserted at the HindIII-EcoRV site of pSSD3. Therefore, the present protein is considered to be a type-II membrane protein. The in vitro translation resulted in the formation of a translation product of 20 kDa that was almost consistent with the molecular weight of 17,307 predicted from the ORF.

The search of the protein data base using the amino acid sequence of the present protein revealed that the protein was analogous to the human early activation antigen CD69 (SWISS-PROT Accession No. Q07108). Table 8 indicates the comparison of the amino acid sequences between the human protein of the present invention (HP) and the human early activation antigen CD69 (CD). - represents a gap, * represents an amino acid residue identical to that in the protein of the present invention, and . represents an amino acid residue analogous to that in the protein of the present invention. The both proteins possessed a homology of 36.6% in the C-terminal region of 112 amino acid residues.

TABLE 8

| | | |
|---|---|---|
| HP | MMTKHKKCFI | |
| CD | MSSENCFVAENSSLHPESGQENDATSPHFSTRHECSFQVPVLCAVMNVVFITILIIALIA | |
| HP | IVQVLITTNIITLIVKLTRDSQSLCPYDWTGFQNKCYYFSKEEGDWNSSKYNCSTQHADL | |
| | * *. **.*.*.***..*. . .*.*.. .**.. *.* | |
| CD | LSVGQYNCPGQYTFSMPSDSHVSSCSEDWVGYQRKCYFISTVKRSWTSAQNACSEHGATL | |
| HP | TIIDNIEEKNFLRRYKCSSDHWIGLKMAKLNRTGQWDGATFTKSFGMRGSEGCAYLSDDG | * .* .*.. *.. **. *. .*.... |
| | ..**. ..****.** ...**.***.. ... .* .* .*.. *.. **.*..*.... | |
| CD | AVIDSEKDMNFLKRYAGREEHWVGLKKEPGHPWKWSNGKEFNNWFNVTGSDKCVFLKNTE | |
| HP | AATARCYTERKWTCRKRIH | |
| | ... * .. ***.* | |
| CD | VSSMECKKNLYWECNKPYK | |

Furthermore, the search of GenBank using the base sequence of the present cDNA revealed that there existed some ESTs possessing the homology of 90% or more (for example, Accession No. H11808), but many sequences are not distinct and the same ORF as that in the present cDNA was not identified.

The human early activation antigen CD69 is a glycoprotein that appears on the surface of activated lymphocytes and a member of the C-type lectin super-family [Hamann, J. et al., J. Immunol. 150: 4920-4927 (1993)]. Since these membrane antigens are expressed specifically in some specific cells, an antibody that is prepared so as to bind to this antigen is applicable for a variety of diagnoses and as a carrier for the drug delivery. Furthermore, cells in which such a membrane antigen is expressed by transduction of the membrane antigen gene are applicable to the detection of the corresponding ligand.

<HP10122> (Sequence Number 12, 37, 62)

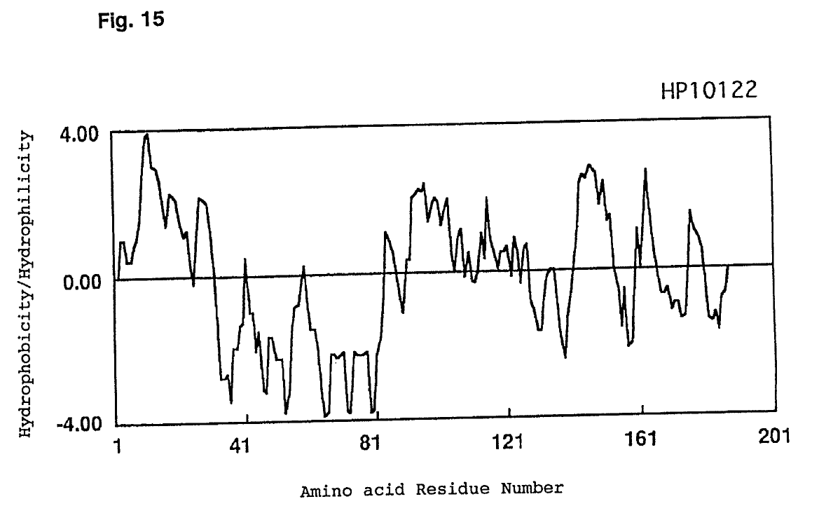

Determination of the whole base sequence for the cDNA insert of clone HP10122 obtained from the human stomach cancer cDNA libraries revealed the structure consisting of a 5′-non-translation region of 138 bp, an ORF of 567 bp, and a 3′-non-translation region of 481 bp. The ORF codes for a protein consisting of 188 amino acid residues with 2 transmembrane domains. FIG. 15 depicts the hydrophobicity/hydrophilicity profile of the present protein obtained by the Kyte-Doolittle method. The in vitro translation resulted in the formation of a translation product of 22 kDa that was almost consistent with the molecular weight of 21,175 predicted from the ORF.

The search of the protein data base using the amino acid sequence of the present protein revealed that the protein was not analogous to any of known proteins. Furthermore, the search of GenBank using the base sequence of the present cDNA revealed that there existed some ESTs possessing the homology of 90% or more (for example, Accession No. T80360), but many sequences were not distinct and the same ORF as that in the present cDNA was not identified.

<HP10136> (Sequence Number 13, 38, 63)

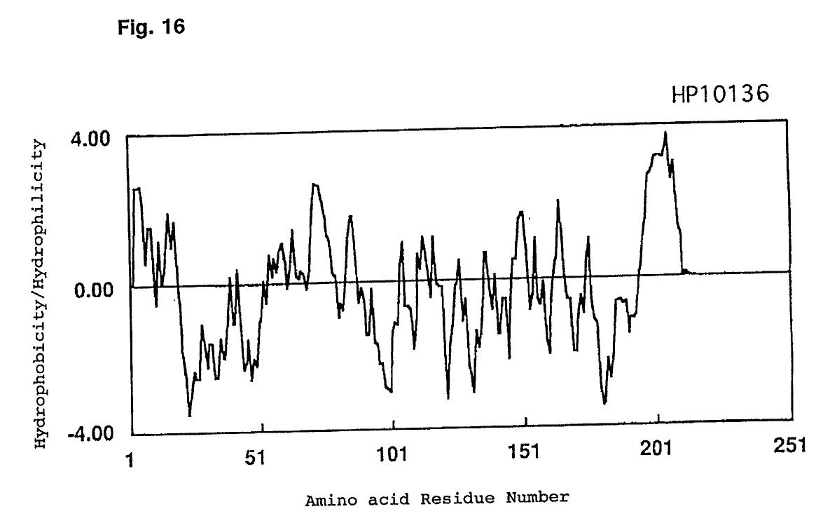

Determination of the whole base sequence for the cDNA insert of clone HP10136 obtained from the human lymphoma cell line U937 cDNA libraries revealed the structure consisting of a 51-non-translation region of 81 bp, an ORF of G48 bp, and a 3′-non-translation region of 680 bp. The ORF codes for a protein consisting of 215 amino acid residues with one transmembrane domain at the C-terminal. FIG. 16 depicts the hydrophobicity/hydrophilicity profile of the present protein obtained by the Kyte-Doolittle method. The in vitro translation resulted in the formation of a translation product of 28 kDa that was almost consistent with the molecular weight of 24,740 predicted from the ORF.

The search of the protein data base using the amino acid sequence of the present protein revealed that the protein was analogous to the baker's yeast protein transport protein SLY2 (SWISS-PROT Accession No. P22214). Table 9 indicates the comparison of the amino acid sequences between the human protein of the present invention (HP) and the baker's yeast protein transport protein SLY2 (SC). - represents a gap, * represents an amino acid residue identical to that in the protein of the present invention, and . represents an amino acid residue analogous to that in the protein of the present invention. The both proteins possessed a homology of 36.1% in the entire regions.

TABLE 9

| | | |
|---|---|---|
| HP | MVLLTMIARVADGLPLAASMQEDEQSGRDLQQYQSQAKQLFRKLNEQSPTRCTLEAGAMT | |
| | *. *.* * ***** .*........ * . . ..* . ..*. **.* ***.*... | |
| SC | MIKSTLIYRE-DGLPLCTSVDNENDPS--LFEQKQKVKIVVSRLTPQSATEATLESGSFE | |
| HP | FHYIIEQGVCYLVLCEAAFPKKLAFAYLEDLHSEFDEQHGKKVPTVS-RPYSFIEFDTFI | |
| | .**. . * * *.**...*..***.**.*. **.. ... *. . *** *..**.*. | *. . *** *. .**.*. |
| SC | IHYLKKSMVYYFVICESGYPRNLAFSYLNDIAQEFEHSFANEYPKPTVRPYQFVNFDNFL | |
| HP | QKTKKLYIDSRARRNLGSINTELQDVQRIMVANIEEVLQRGEALSALDSKANNLSSLSKK | |
| | *.*** * *... **. .* ** .*..** ***..* **..*. .......*.. **. | *.. **. |
| SC | QMTKKSYSDKKVQDNLDQLNQELVGVKQIMSKNIEDLLYRGDSLDKMSDMSSSLKETSKR | |
| HP | YRQDAKYLNMRSTYAKLAAVAVFFIMLIVYVRFWWL | |
| | **..*. .*. .. *.. *. ...*.. ***. | |
| SC | YRKSAQKINFDLLISQYAPI-VIVAFFFVFL-FWWIFLK | |

Furthermore, the search of GenBank using the base sequence of the present cDNA revealed that there existed some ESTs possessing the homology of 90% or more (for example, Accession No. R80136), but they were shorter than the present cDNA and any molecule containing the initiation codon was not identified.

The baker's yeast protein transport protein SLY2 has been known to be essential for endoplasmic reticulum-to-Golgi protein transport and to be also associated with the control of the cell cycle [Dascher, C. et al., Mol. Cell. Biol. 11: 872-885 (1991)]. Therefore, the cDNA of the present invention can be utilized for the production of the present protein as well as for the diagnosis and the treatment of diseases caused by the abnormality of the present protein.

<HP10175> (Sequence Number 14, 39, 64)

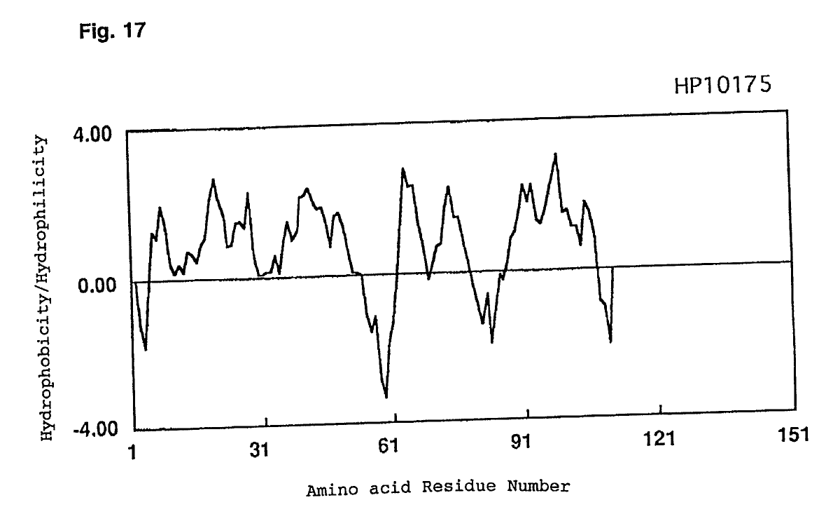

Determination of the whole base sequence for the cDNA insert of clone HP10175 obtained from the human stomach cancer cDNA libraries revealed the structure consisting of a 5′-non-translation region of 173 bp, an ORF of 339 bp, and a 3′-non-translation region of 462 bp. The ORF codes for a protein consisting of 112 amino acid residues with 4 transmembrane domains. FIG. 17 depicts the hydrophobicity/hydrophilicity profile of the present protein obtained by the Kyte-Doolittle method. The result of the in vitro translation resulted in the formation of a translation product of 13 kDa that was almost consistent with the molecular weight of 11,564 predicted from the ORF.

The search of the protein data base using the amino acid sequence of the present protein revealed that the protein was not analogous to any known proteins. Furthermore, the search of GenBank using the base sequence of the present cDNA revealed that there existed some ESTs possessing the homology of 90% or more (for example, Accession No. W52852), but many sequences were not distinct and the same ORF as that in the present cDNA was not identified.

<HP10179> (Sequence Number 15, 40, 65)

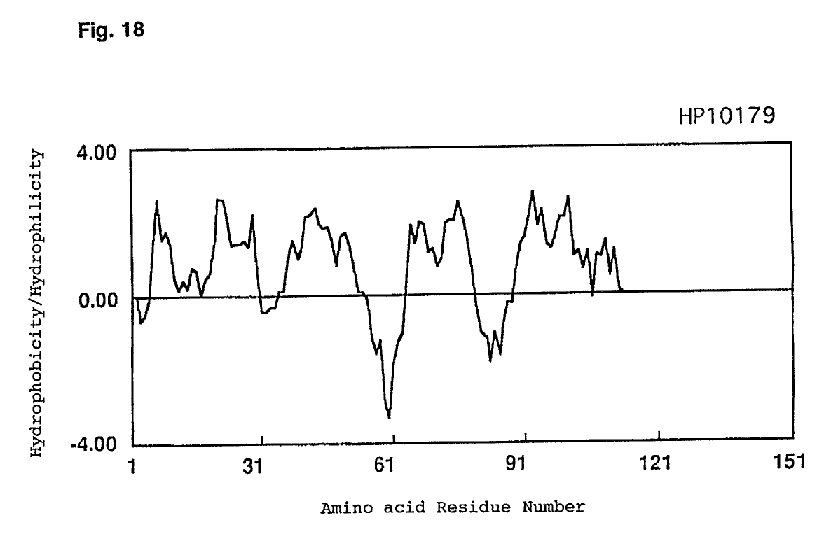

Determination of the whole base sequence for the cDNA insert of clone HP10179 obtained from the human epidermoid carcinoma cell line KB cDNA libraries revealed the structure consisting of a 5′-non-translation region of 121 bp, an ORF of 345 bp, and a 3′-non-translation region of 459 bp. The ORF codes for a protein consisting of 114 amino acid residues with 4 transmembrane domains. FIG. 18 depicts the hydrophobicity/hydrophilicity profile of the present protein obtained by the Kyte-Doolittle method. The in vitro translation resulted in the formation of a translation product of 14 kDa that was almost consistent with the molecular weight of 12,078 predicted from the ORF.

The search of the protein data base using the amino acid sequence of the present protein revealed that the protein was not analogous to any known proteins. However, this protein was analogous to the protein encoded by the cDNA clone Hp 10175 of the present invention. Table 10 indicates the comparison of the amino acid sequences between the protein encoded by HP 10179 and the protein encoded by HP 10175. - represents a gap, * represents an amino acid residue identical to that in the protein of the present invention, and . represents an amino acid residue analogous to that in the protein of the present invention. The both proteins possessed a homology of 80.8% in the entire regions.

TABLE 10

| | |
|---|---|
| 179 | MEKPLFPLVPLHWFGFGYTALVVSGGIVGYVKTGSVPSLAAGLLFGSLAGLGAYQLYQDP |
| | ..**********.***.****.****.*********************** *** |
| 175 | MQDTQSVVPLBWFGFGYAALVASGGIIGYVKAGSVPSLAAGLLFGSLAGLGAYQLSQDP |
| 179 | RNVWGFLAATSVTFVGVMGMRSYYYGKFMPVGLIAGASLLMAAKVGVRMLMTSD |
| | **** ** *** *..*.**** *. *****.**********.*****.*. |
| 175 | RNVWVFL-ATSCTLAGIMGMRFYHSGKFMPAGLIAGASLLMVAKVGVSMFNRPH |

Furthermore, the search of GenBank using the base sequence of the present cDNA revealed that there existed some ESTs possessing the homology of 90% or more (for example, Accession No. N55991), but many sequences were not distinct and the same ORF as that in the present cDNA was not identified.

<HP10196> (Sequence Number 16, 41, 66)

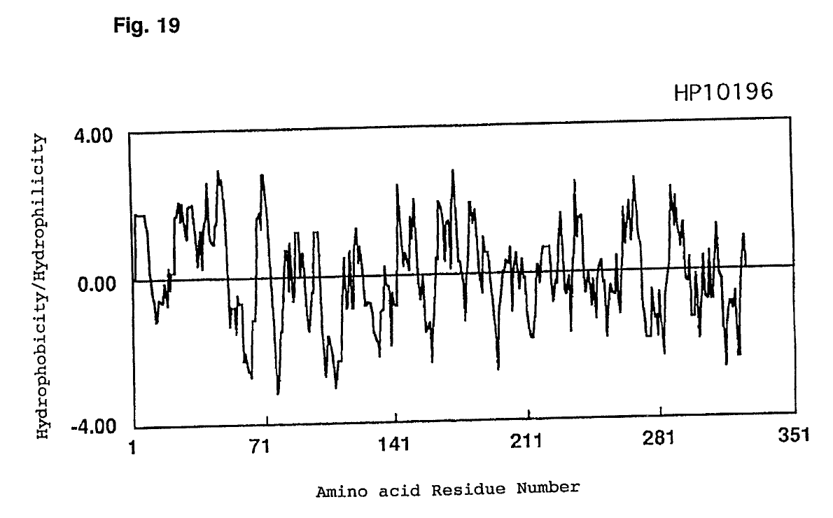

Determination of the whole base sequence for the cDNA insert of clone HP10196 obtained from the human fibrosarcoma cell line HT-1080 cDNA libraries revealed the structure consisting of a 5′-non-translation region of 9 bp, an ORF of 984 bp, and a 3′-non-translation region of 122 bp. The ORF codes for a protein consisting of 327 amino acid residues with one transmembrane domain at the N-terminal. FIG. 19 depicts the hydrophobicity/hydrophilicity profile of the present protein obtained by the Kyte-Doolittle method. It was indicated that the present protein remained in the membrane from the observation that the urokinase secretion was not identified upon transduction into the COS7 cells of an expression vector in which a HindIII-Bg1II fragment (after the Klenow treatment) containing a cDNA fragment encoding the N-terminal 162 amino acid residues in the present protein was inserted at the HindIII-EcoRV site of pSSD3. Therefore, the present protein is considered to be a type-II membrane protein. The in vitro translation resulted in the formation of a translation product of 37 kDa that was almost consistent with the molecular weight of 36,163 predicted from the ORF.

The search of the protein data base using the amino acid sequence of the present protein revealed that the protein was not analogous to any known proteins. Furthermore, the search of GenBank using the base sequence of the present cDNA revealed that there existed some ESTs possessing the homology of 90% or more (for example, Accession No. T17026), but they were shorter than the present cDNA and any molecule containing the initiation codon was not identified.

<HP10235> (Sequence Number 17, 42, 67)

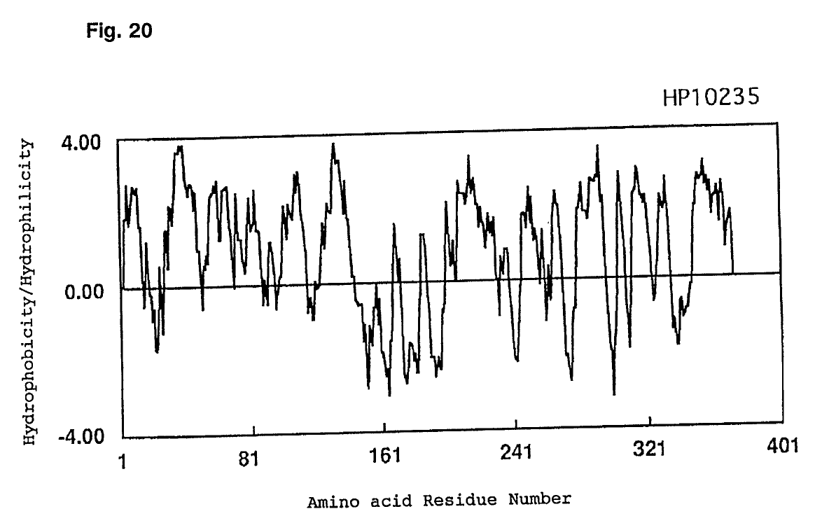

Determination of the whole base sequence for the cDNA insert of clone HP10235 obtained from the human fibrosarcoma cell line HT-1080 cDNA libraries revealed the structure consisting of a 5′-non-translation region of 5 bp, an ORF of 1122 bp, and a 3′-non-translation region of 594 bp. The ORF codes for a protein consisting of 373 amino acid residues with 11 transmembrane domains. FIG. 20 depicts the hydrophobicity/hydrophilicity profile of the present protein obtained by the Kyte-Doolittle method. The in vitro translation did not reveal the formation of distinct bands and revealed the formation of smeary bands at the high-molecular-weight position.

The search of the protein data base using the amino acid sequence of the present protein revealed that the protein was analogous to the human nucleolar protein HNP36 (EMBL Accession No. X86681). Table 11 indicates the comparison of the amino acid sequences between the human protein of the present invention (HP) and the human nucleolar protein HNP36 (NP). - represents a gap, * represents an amino acid residue identical to that in the protein of the present invention, and . represents an amino acid residue analogous to that in the protein of the present invention. The both proteins possessed a homology of 45.3% in the entire regions.

TABLE 11

| | | |
|---|---|---|
| HP | MTLCAMLPLLLFTYLNSFLHQRIPQSVRILGSLVAILLVFLITAILVKVQLDALPFFVIT | |
| HP | MIKIVLINSFGAILQGSLFGLAGLLPASYTAPIMSGQGLAGFFASVAMICAIASGSELSE | |
| | * .. .****.*.******* * .*..*.. ..*******.**..**. ..*** . .. | |
| NP | MASVCFINSFSAVLQGSLFGQLGTMPSTYSTLSGQGLAGIFAALAMLLSMASGVDAET | |
| HP | SAFGYFITPYVGILMSIVCYLSLPHLKFARYYLANKSSQAQAQELETKAELLQSDENGIP | |
| | **.*****... *...*.***.**.*.* *** . * . .** ** .*. .*. | |
| NP | SALGYFITPYVGILMSIVCYLSLPHLKFARYYLANKSSQAQAQELETKAELLQSDENGIP | |
| HP | --PRAGKEESGVSV---SNSQPTNESHSIK----AILKNTSVLAFSVCFIFTITIGMFPA | |
| | *. . .... ..*.*.. ..* ....* . *... ..**.*...*** | *.* |
| NP | SSPQKVALTLDLDLEKEPESEPDEPQKPGKPSVFTVFQKIWLTALCLVLVFTVTLSVFPA | |
| HP | VTVEVKSSIAGSSTWERYFIPVSCFLTFNIFDWLGRSLTAVFMWPGKDSRWLPSLVLARL | |
| | .*. *.** .....*...* *..*** ***.*******. *.**..*** ** ** *. | |
| NP | ITAMVTSS-TSPGKWSQFFNPICCFLLFNINDWLGRSLTSYFLWPDEDSRLLPLLVCLRF | |
| HP | VFVPLLLLCNIKPRRYLTVVFEHDAWFIFFMAAFAFSNGYLASLCMCFGPKKVPAEAET | |
| | .****..**.. .*. *...* .**.** ** ** *****.** **..*..* * * *. | |
| NP | LFVPLFMLCHVPQRSRLPILFPQDAYFITFMLLFAVSNGYLVSLTMCLAPRQVLPHEREV | |
| HP | AGAIMAFFLCLGLALGAVFSFLFRAIV | |
| | ***.*.*** ***. ** .****.*.. | |
| NP | AGALMTFFLALCLSCGASLSFLFKALL | |

Furthermore, the search of GenBank using the base sequence of the present cDNA revealed that there existed some ESTs possessing the homology of 90% or more (for example, Accession No. R57372), but it can not be assessed whether these ESTs with partial sequences code for the same protein as the protein of the present invention.

The human nucleolar protein HNP36 has been found as a gene product that plays a role in the growth and multiplication of cells [Williams, J. B. & Lanahan, A. A., Biochem. Biophys. Res. Commun. 213: 325-333 (1995)]. Accordingly, the protein of present invention, which is homologous to said protein, is considered to be a housekeeping protein essential to the growth and multiplication of cells and thereby can be utilized for the diagnosis and the treatment of diseases caused by the abnormality of the present protein.

<HP10297> (Sequence Number 18, 43, 68)

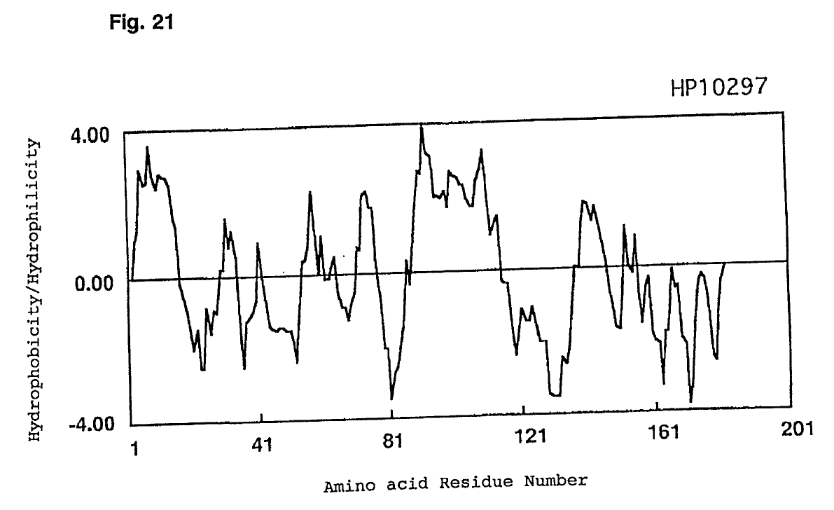

Determination of the whole base sequence for the cDNA insert of clone HP10297 obtained from the human stomach cancer cDNA libraries revealed the structure consisting of a 5′-non-translation region of 62 bp, an ORF of 552 bp, and a 3′-non-translation region of 890 bp. The ORF codes for a protein consisting of 183 amino acid residues with a signal sequence at the N-terminal and one internal transmembrane domain. Therefore, the present protein is considered to be a type-I membrane protein. FIG. 21 depicts the hydrophobicity/hydrophilicity profile of the present protein obtained by the Kyte-Doolittle method. The in vitro translation resulted in the formation of a translation product of 24 kDa that was almost consistent with the molecular weight of 20,574 predicted from the ORF.

The search of the protein data base using the amino acid sequence of the present protein revealed that the protein was not analogous to any known proteins. Furthermore, the search of GenBank using the base sequence of the present cDNA revealed that there existed some ESTs possessing the homology of 90% or more (for example, Accession No. R47823), but many sequences are not distinct and the same ORF as that in the present cDNA was not identified.

<HP10299> (Sequence Number 19, 44, 69)

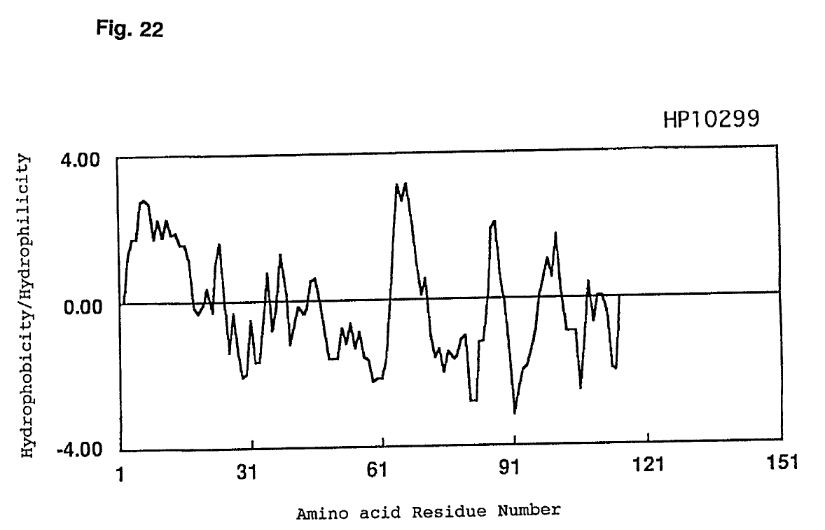

Determination of the whole base sequence for the cDNA insert of clone HP10299 obtained from the human stomach cancer cDNA libraries revealed the structure consisting of a 5′-non-translation region of 92 bp, an ORF of 351 bp, and a 3′-non-translation region of 89 bp. The ORF codes for a protein consisting of 116 amino acid residues with one transmembrane domain at the N-terminal. FIG. 22 depicts the hydrophobicity/hydrophilicity profile of the present protein obtained by the Kyte-Doolittle method. It was indicated that the present protein remained in the membrane from the observation that the urokinase secretion was not identified upon transduction into the COS7 cells of an expression vector in which a HindIII-VspI fragment (after the Klenow treatment) containing a cDNA fragment encoding the N-terminal 65 amino acid residues in the present protein was inserted at the HindIII-PmaCI site of pSSD3. Therefore, the present protein is considered to be a type-II membrane protein. The in vitro translation resulted in the formation of a translation product of 13 kDa that was almost consistent with the molecular weight of 12,498 predicted from the ORF.

The search of the protein data base using the amino acid sequence of the present protein revealed that the protein was analogous to the baker's yeast hypothetical membrane protein of 16.5 kDa (SWISS-PROT Accession No. P42834). Table 12 indicates the comparison of the amino acid sequences between the human protein of the present invention (HP) and the baker's yeast hypothetical membrane protein of 16.5 kDa (SC). - represents a gap, * represents an amino acid residue identical to that in the protein of the present invention, and . represents an amino acid residue analogous to that in the protein of the present invention. The both proteins possessed a homology of 53.0% in the C-terminal region of 66 amino acid residues.

TABLE 12

| | |
|---|---|
| HP | MASTVVAVGLTIAAAGFAGRYVL |

QAMKHMEPQVKQVF

| | |
|---|---|
| SC | MVLPIIIGLGVTMVALSVKSGLNAWTVYKTLSPLTIAKLNNIRIENP |

TAGYRDALKFKSS

| | |
|---|---|
| HP | QSLPKSAFSGGYYRGGFEPKMTKREAALILGVSP----TANKGKIRD |
| | *.***.*.**..** ***..*. .. ... |

AHRRIMLLNHPDK
**. *. ****.

| | |
|---|---|
| SC | LIDEELKNRLNQYQGGFAPRMTEPEALLILDISAREINHLDEKLLKK |

KHRKAMVRNHPDR

| | |
|---|---|
| HP | GGSPYIAAKINEAKDLLEGQAKK |
| | *****.********..** |
| SC | GGSPYMAAKINEAKEVLERSVLLRKR |

Furthermore, the search of GenBank using the base sequence of the present cDNA revealed that there existed some ESTs possessing the homology of 90% or more (for example, Accession No. R27748), but many sequences were not distinct and the same ORF as that in the present cDNA was not identified.

<HP10301> (Sequence Number 20, 45, 70)

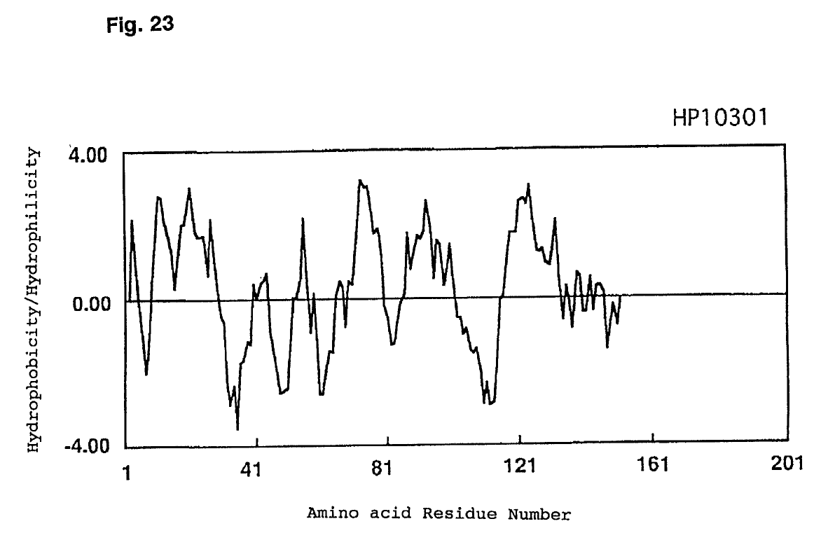

Determination of the whole base sequence for the cDNA insert of clone HP10301 obtained from the human epidermoid carcinoma cell line KB cDNA libraries revealed the structure consisting of a 5′-non-translation region of 91 bp, an ORF of 459 bp, and a 3′-non-translation region of 112 bp. The ORF codes for a protein consisting of 152 amino acid residues with four transmembrane domains. FIG. 23 depicts the hydrophobicity/hydrophilicity profile of the present protein obtained by the Kyte-Doolittle method. The in vitro translation resulted in the formation of a translation product of 18 kDa that was almost consistent with the molecular weight of 16,516 predicted from the ORF.

The search of the protein data base using the amino acid sequence of the present protein revealed that the protein was not analogous to any known proteins. Furthermore, the search of GenBank using the base sequence of the present cDNA revealed that there existed some ESTs possessing the homology of 90% or more (for example, Accession No. N28828), but many sequences were not distinct and the same ORF as that in the present cDNA was not identified.

<HP10302> (Sequence Number 21, 46, 71)

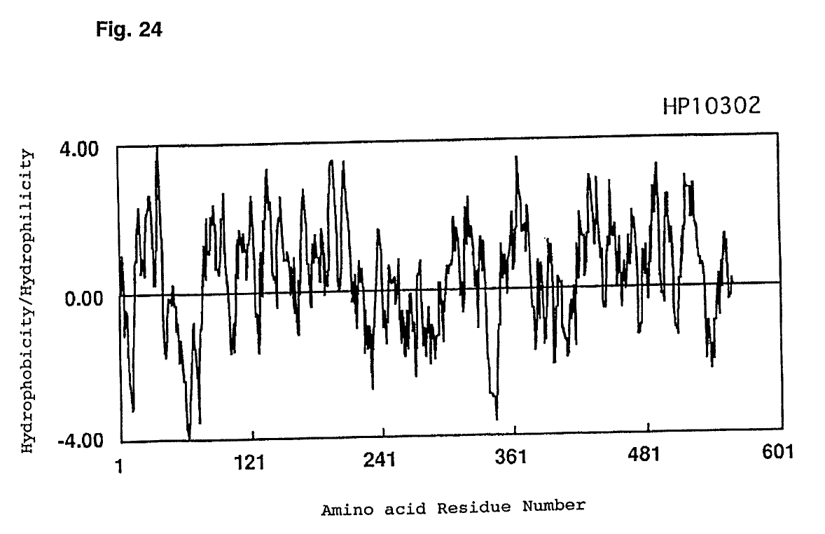

Determination of the whole base sequence for the cDNA insert of clone HP10302 obtained from the human liver cDNA libraries revealed the structure consisting of a 5′-non-translation region of 133 bp, an ORF of 1680 bp, and a 3′-non-translation region of 560 bp. The ORF codes for a protein consisting of 559 amino acid residues with 12 transmembrane domains. FIG. 24 depicts the hydrophobicity/hydrophilicity profile of the present protein obtained by the Kyte-Doolittle method. The in vitro translation did not reveal the formation of distinct bands and revealed the formation of smeary bands at the high-molecular-weight position.

The search of the protein data base using the amino acid sequence of the present protein revealed that the protein was not analogous to any known proteins. Furthermore, the search of GenBank using the base sequence of the present cDNA revealed that there existed some ESTs possessing the homology of 90% or more (for example, Accession No. N72434), but they were shorter than the present cDNA and any molecule containing the initiation codon was not identified.

<HP10304> (Sequence Number 22, 47, 72)

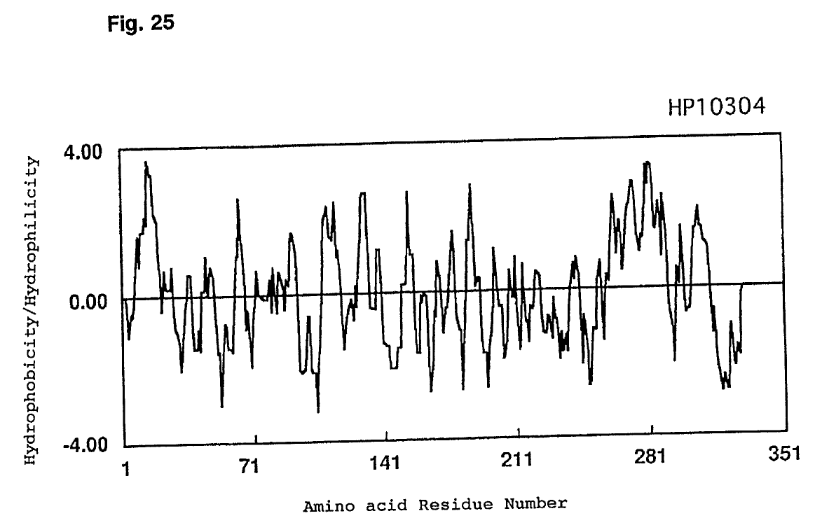

Determination of the whole base sequence for the cDNA insert of clone HP10304 obtained from the human osterosarcoma U-2 OS cDNA libraries revealed the structure consisting of a 5′-non-translation region of 10 bp, an ORF of 993 bp, and a 3′-non-translation region of 313 bp. The ORF codes for a protein consisting of 330 amino acid residues with a signal sequence at the N-terminal and one internal transmembrane domain. Therefore, the present protein is considered to be a type-I membrane protein. FIG. 25 depicts the hydrophobicity/hydrophilicity profile of the present protein obtained by the Kyte-Doolittle method. The in vitro translation resulted in the formation of a translation product of 36 kDa that was almost consistent with the molecular weight of 36,840 predicted from the ORF.

The search of the protein data base using the amino acid sequence of the present protein revealed that the protein was not analogous to any known proteins. Furthermore, the search of GenBank using the base sequence of the present cDNA revealed that there existed some ESTs possessing the homology of 90% or more (for example, Accession No. N26840), but the same ORF as that in the present cDNA was not identified.

<HP10305> (Sequence Number 23, 48, 73)

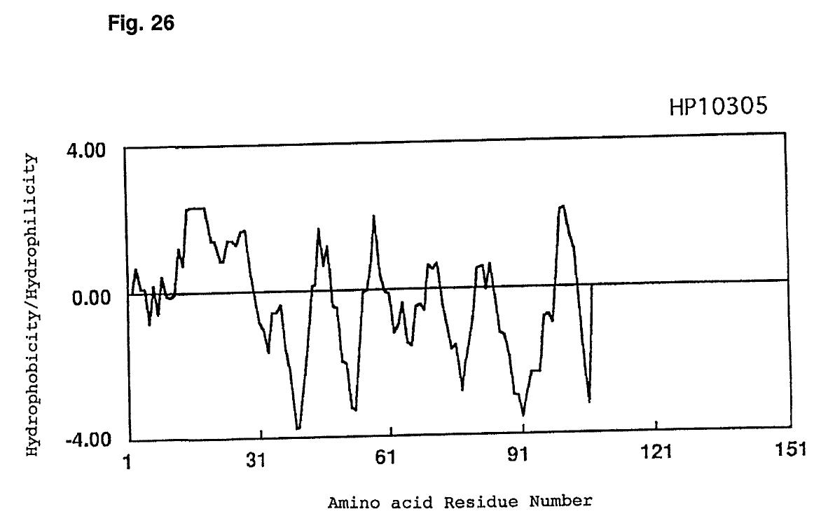

Determination of the whole base sequence for the cDNA insert of clone HP10305 obtained from the human osterosarcoma U-2 OS cDNA libraries revealed the structure consisting of a 5′-non-translation region of 109 bp, an ORF of 327 bp, and a 3′-non-translation region of 457 bp. The ORF codes for a protein consisting of 108 amino acid residues with one transmembrane domain. FIG. 26 depicts the hydrophobicity/hydrophilicity profile of the present protein obtained by the Kyte-Doolittle method. It was indicated that the present protein remained in the membrane from the observation that the urokinase secretion was not identified upon transduction into the COS7 cells of an expression vector in which a HindIII-ApaI fragment (treated with mung-bean nuclease) containing a cDNA fragment encoding the N-terminal 162 amino acid residues in the present protein was inserted at the HindIII-PmaCI site of pSSD3. Therefore, the present protein is considered to be a type-II membrane protein. The in vitro translation resulted in the formation of a translation product of 15 kDa that was almost consistent with the molecular weight of 12,199 predicted from the ORF.

The search of the protein data base using the amino acid sequence of the present protein revealed that the protein was not analogous to any known proteins. Furthermore, the search of GenBank using the base sequence of the present cDNA revealed that there existed some ESTs possessing the homology of 90% or more (for example, Accession No. H02768), but many sequences are not distinct and the same ORF as that in the present cDNA was not identified.

<HP10306> (Sequence Number 24, 49, 74)

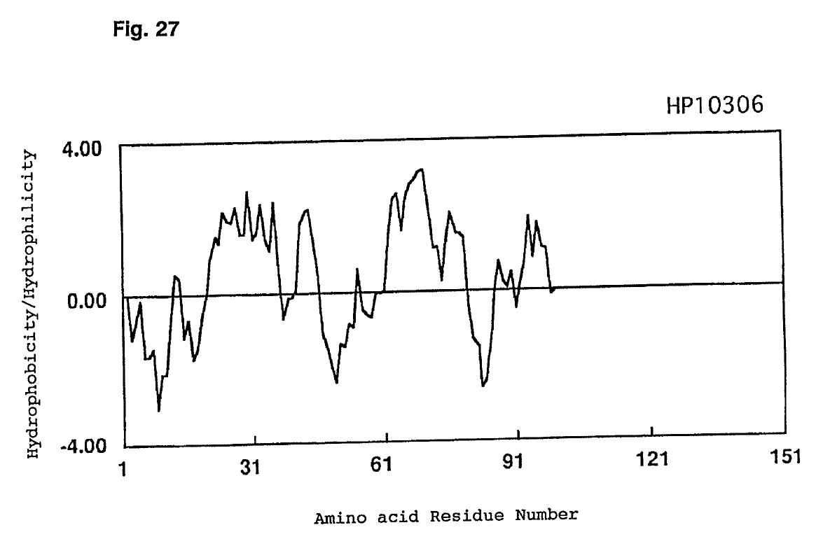

Determination of the whole base sequence for the cDNA insert of clone HP10306 obtained from the human osterosarcoma U-2 OS cDNA libraries revealed the structure consisting of a 5′-non-translation region of 229 bp, an ORF of 306 bp, and a 3′-non-translation region of 155 bp. The ORF codes for a protein consisting of 101 amino acid residues with 2 transmembrane domains. FIG. 27 depicts the hydrophobicity/hydrophilicity profile of the present protein obtained by the Kyte-Doolittle method. The in vitro translation resulted in the formation of a translation product of 14 kDa that was almost consistent with the molecular weight of 12,029 predicted from the ORF.

The search of the protein data base using the amino acid sequence of the present protein revealed that the protein was not analogous to any known proteins. Furthermore, the search of GenBank using the base sequence of the present cDNA revealed that there existed some ESTs possessing the homology of 90% or more (for example, Accession No. H44711), but many sequences are not distinct and the same ORF as that in the present cDNA was not identified.

<HP10328> (Sequence Number 25, 50, 75)

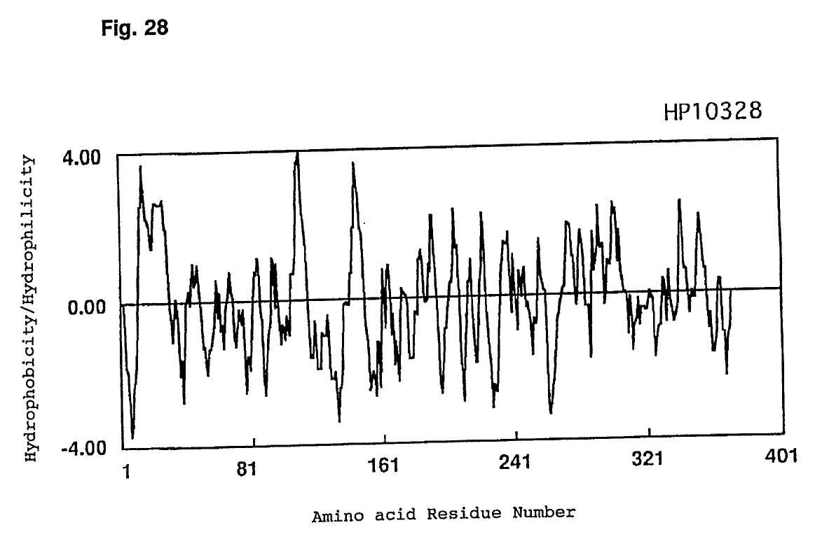

Determination of the whole base sequence for the cDNA insert of clone HP10328 obtained from the human epidermoid carcinoma cell line KB cDNA libraries revealed the structure consisting of a 5′-non-translation region of 117 bp, an ORF of 1119 bp, and a 3′-non-translation region of 950 bp. The ORF codes for a protein consisting of 372 amino acid residues with one transmembrane domain. FIG. 28 depicts the hydrophobicity/hydrophilicity profile of the present protein obtained by the Kyte-Doolittle method. It was indicated that the present protein remained in the membrane from the observation that the urokinase secretion was not identified upon transduction into the COS7 cells of an expression vector in which a HindIII-PmaCI fragment (treated with mung-bean nuclease) containing a cDNA fragment encoding the N-terminal 129 amino acid residues in the present protein was inserted at the HindIII-SmaI site of pSSD3. Therefore, the present protein is considered to be a type-II membrane protein. The in vitro translation resulted in the formation of a translation product of 41 kDa that was almost consistent with the molecular weight of 42,514 predicted from the ORF.

The search of the protein data base using the amino acid sequence of the present protein revealed that the protein was analogous to the Drosophila neurological secretory signal protein (GenBank Accession No. U41449). Table 13 indicates the comparison of the amino acid sequences between the human protein of the present invention (HP) and the Drosophila neurological secretory signal protein (DM). - represents a gap, * represents an amino acid residue identical to that in the protein of the present invention, and . represents an amino acid residue analogous to that in the protein of the present invention. The both proteins possessed a homology of 38.6% in the middle region of 202 amino acid residues.

TABLE 13

| | |
|---|---|
| HP | MKYLRHRRPNATLILAIGAFTLLLFSLLVSPPTCKVQEQPPAIPEALAWPTPPTRPAPAP |
| DM | MQSKHRKLLLRCLLVLPLILLVDYCGLLTHL |
| HP | CHANTSMVTHPDFATQPQHVQNFLLYRHCRHFPLLQDVPPSKCAQPVFLLLVIKSSPSNY |
| | **. * ..***. .* |
| DM | HELNFERFHYPLNDDTGSGSASSGLDKFAYLRVPSFTAEVPVDQPARLTMLIKSAVGNS |
| HP | VRRELLRRTWGRERKVRGLQLRLLFLVGTASNPHEARKVNRLLELEAQTHGDILQWDFHD |
| | *** .***** * . ....** **.***... ...*.. *...****** ** * |
| DM | RRREAIRRTWGYEGRFSDVHLRRVFLLGTAEDS--EKDVAW----ESREHGDILQADFTD |
| HP | SFFNLTLKQVLFLQWQETRCANASFVLNGDDDVFAHTDNMVFYL----QDHDPGRHLFVG |
| | ..** *** .* ..*.... ....* * *** .. ..*.. .* *.*.*. **.* |
| DM | AYFNNTLKTMLGMRWASEQFNRSEFYLFVDDDYYVSAKNVLKFLGRGRQSHQPE-LLFAG |
| HP | QLIQNVGPIRAFWSKYYVPEVVTQNERYPPYCGGGGFLLSRFTAAALRRAAHVLDIFPID |
| | ...* ..*.* .**.**. . .*.*** ..*.**. . .* *. * .*..* |
| DM | HVFQ-TSPLRHKFSKWYVSLEEYPFDRWPPYVTAGAFILSQKALRQLYAASVHLPLFRFD |
| HP | DVFLGMCLELEGLKPASHSGIRTSGVRAPSQHLSSFDPCFYRDLLLVHRFLPYEMLLMWD |
| | **.**. |
| DM | DVYLGIVALKAGISLQHCDDFRFHRPAYKGPDSYSSVIASHEFGDPEEMTRVWNECRSAN |
| HP | ALNQPNLTCCNQTQIY |
| DM | YA |

Furthermore, the search of GenBank using the base sequence of the present cDNA revealed that there existed some ESTs possessing the homology of 90% or more (for example, Accession No. R75815), but they were shorter than the present cDNA and any molecule containing the initiation codon was not identified.

The present invention provides human proteins having transmembrane domains, cDNAs encoding said proteins and eykaryotic cells expressing said cDNA. All of the proteins of the present invention are putative proteins controlling the proliferation and differentiation of the cells, because said proteins exist on the cell membrane. Therefore, the proteins of the present invention can be used as pharmaceuticals or as antigens for preparing antibodies against said proteins. Furthermore, said DNAs can be used for the expression of large amounts of said proteins. The cells expressing large amounts of membrane proteins with transfection of these membrane protein genes can be applied to the detection of the corresponding ligands, the screening of novel low-molecular medicines, and so on.

In addition to the activities and uses described above, the polynucleotides and proteins of the present invention may exhibit one or more of the uses or biological activities (including those associated with assays cited herein) identified below. Uses or activities described for proteins of the present invention may be provided by administration or use of such proteins or by administration or use of polynucleotides encoding such proteins (such as, for example, in gene therapies or vectors suitable for introduction of DNA).

### Research Uses and Utilities

The polynucleotides provided by the present invention can be used by the research community for various purposes. The polynucleotides can be used to express recombinant protein for analysis, characterization or therapeutic use; as markers for tissues in which the corresponding protein is preferentially expressed (either constitutively or at a particular stage of tissue differentiation or development or in disease states); as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; as a probe to “'subtract-out” known sequences in the process of discovering other novel polynucleotides; for selecting and making oligomers for attachment to a “gene chip” or other support, including for examination of expression patterns; to raise anti-protein antibodiesusing DNA immunization techniques; and as an antigen to raise anti-DNA antibodies or elicit another immune response. Where the polynucleotide encodes a protein which binds or potentially binds to another protein (such as, for example, in a receptor-ligand interaction), the polynucleotide can also be used in interaction trap assays (such as, for example, that described in Gyuris et al., Cell 75:791-803 (1993)) to identify polynucleotides encoding the other protein with which binding occurs or to identify inhibitors of the binding interaction.

The proteins provided by the present invention can similarly be used in assay to determine biological activity, including in a panel of multiple proteins for high-throughput screening; to raise antibodies or to elicit another immune response; as a reagent (including the labeled reagent) in assays designed to quantitatively determine levels of the protein (or its receptor) in biological fluids; as markers for tissues in which the corresponding protein is preferentially expressed (either constitutively or at a particular stage of tissue differentiation or development or in a disease state); and, of course, to isolate correlative receptors or ligands. Where the protein binds or potentially binds to another protein (such as, for example, in a receptor-ligand interaction), the protein can be used to identify the other protein with which binding occurs or to identify inhibitors of the binding interaction. Proteins involved in these binding interactions can also be used to screen for peptide or small molecule inhibitors or agonists of the binding interaction.

Any or all of these research utilities are capable of being developed into reagent grade or kit format for commercialization as research products.

Methods for performing the uses listed above are well known to those skilled in the art. References disclosing such methods include without limitation “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

### Nutritional Uses

Polynucleotides and proteins of the present invention can also be used as nutritional sources or supplements. Such uses include without limitation use as a protein or amino acid supplement, use as a carbon source, use as a nitrogen source and use as a source of carbohydrate. In such cases the protein or polynucleotide of the invention can be added to the feed of a particular organism or can be administered as a separate solid or liquid preparation, such as in the form of powder, pills, solutions, suspensions or capsules. In the case of microorganisms, the protein or polynucleotide of the invention can be added to the medium in or on which the microorganism is cultured.

### Cytokine and Cell Proliferation/Differentiation Activity

A protein of the present invention may exhibit cytokine, cell proliferation (either inducing or inhibiting) or cell differentiation (either inducing or inhibiting) activity or may induce production of other cytokines in certain cell populations. Many protein factors discovered to date, including all known cytokines, have exhibited activity in one or more factor dependent cell proliferation assays, and hence the assays serve as a convenient confirmation of cytokine activity. The activity of a protein of the present invention is evidenced by any one of a number of routine factor dependent cell proliferation assays for cell lines including, without limitation, 32D, DA2, DA1G, T10, B9, B9/11, BaF3, MC9/G, M+ (preB M+), 2E8, RB5, DA1, 123, T1165, HT2, CTLL2, TF-1, Mo7e and CMK.

The activity of a protein of the invention may, among other means, be measured by the following methods:

Assays for T-cell or thymocyte proliferation include without limitation those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Bertagnolli et al., J. Immunol. 145:1706-1712, 1990; Bertagnolli et al., Cellular Immunology 133:327-341, 1991; Bertagnolli, et al., J. Immunol. 149:3778-3783, 1992; Bowman et al., J. Immunol. 152: 1756-1761, 1994.

Assays for cytokine production and/or proliferation of spleen cells, lymph node cells or thymocytes include, without limitation, those described in: Po lyclonal T cell stimulation, Kruisbeek, A. M. and Shevach, E. M. In Current Protocols in Immunology. J.E.e.a. Coligan eds. Vol 1 pp. 3.12.1-3.12.14, John Wiley and Sons, Toronto. 1994; and Measurement of mouse and human Interferon γ, Schreiber, R. D. In Current Protocols in Immunology. J.E.e.a. Coligan eds. Vol 1 pp. 6.8.1-6.8.8, John Wiley and Sons, Toronto. 1994.

Assays for proliferation and differentiation of hematopoietic and lymphopoietic cells include, without limitation, those described in: Measurement of Human and Murine Interleukin 2 and Interleukin 4, Bottomly, K., Davis, L. S. and Lipsky, P. E. In Current Protocols in Immunology. J.E.e.a. Coligan eds. Vol 1 pp. 6.3.1-6.3.12, John Wiley and Sons, Toronto. 1991; deVries et al., J. Exp. Med. 173:1205-1211, 1991; Moreau et al., Nature 336:690-692, 1988; Greenberger et al., Proc. Natl. Acad. Sci. U.S.A. 80:2931-2938, 1983; Measurement of mouse and human Interleukin 6 -Nordan, R. In Current Protocols in Immunology. J.E.e.a. Coligan eds. Vol 1 pp. 6.6.1-6.6.5, John Wiley and Sons, Toronto. 1991; Smith et al., Proc. Natl. Acad. Sci. U.S.A. 83:1857-1861, 1986; Measurement of human Interleukin 11—Bennett, F., Giannotti, J., Clark, S. C. and Turner, K. J. In Current Protocols in Immunology. J.E.e.a. Coligan eds. Vol 1 pp. 6.15.1 John Wiley and Sons, Toronto. 1991; Measurement of mouse and human Interleukin 9—Ciarletta, A., Giannotti, J., Clark,S. C. and Turner, K. J. In Current Protocols in Immunology. J.E.e.a. Coligan eds. Vol 1 pp. 6.13.1, John Wiley and Sons, Toronto. 1991.

Assays for T-cell clone responses to antigens (which will identify, among others, proteins that affect APC-T cell interactions as well as direct T-cell effects by measuring proliferation and cytokine production) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function; Chapter 6, Cytokines and their cellular receptors; Chapter 7, Immunologic studies in Humans); Weinberger et al., Proc. Natl. Acad. Sci. USA 77:6091-6095, 1980; Weinberger et al., Eur. J. Immun. 11:405-411, 1981; Takai et al., J. Immunol. 137:3494-3500, 1986; Takai et al., J. Immunol. 140:508-512, 1988.

### Immune Stimulating or Suppressing Activity

A protein of the present invention may also exhibit immune stimulating or immune suppressing activity, including without limitation the activities for which assays are described herein. A protein may be useful in the treatment of various immune deficiencies and disorders (including severe combined immunodeficiency (SCID)), e.g., in regulating (up or down) growth and proliferation of T and/or B lymphocytes, as well as effecting the cytolytic activity of NK cells and other cell populations. These immune deficiencies may be genetic or be caused by viral (e.g., HIV) as well as bacterial orfungal infections, or may result from autoimmune disorders. More specifically, infectious diseases causes by viral, bacterial, fungal or other infection may be treatable using a protein of the present invention, including infections by HIV, hepatitis viruses, herpesviruses, mycobacteria, Leishmania spp., malaria spp. and various fungal infections such as candidiasis. Of course, in this regard, a protein of the present invention may also be useful where a boost to the immune system generally may be desirable, i.e., in the treatment of cancer.

Autoimmune disorders which may be treated using a protein of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitis, myasthenia gravis, graft-versus-host disease and autoimmune inflammatory eye disease. Such a protein of the present invention may also to be useful in the treatment of allergic reactions and conditions, such as asthma (particularly allergic asthma) or other respiratory problems. Other conditions, in which immune suppression is desired (including, for example, organ transplantation), may also be treatable using a protein of the present invention.

Using the proteins of the invention it may also be possible to immune responses, in a number of ways. Down regulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or anergy in T cells, is distinguishable from immunosuppression in that it is generally antigen-specific and persists after exposure to the tolerizing agent has ceased. Operationally, tolerance can be demonstrated by the lack of a T cell response upon reexposure to specific antigen in the absence of the tolerizing agent.

Down regulating or preventing one or more antigen functions (including without limitation B lymphocyte antigen functions (such as , for example, B7)), e.g., preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a molecule which inhibits or blocks interaction of a B7 lymphocyte antigen with its natural ligand(s) on immune cells (such as a soluble, monomeric form of a peptide having B7-2 activity alone or in conjunction with a monomeric form of a peptide having an activity of another B lymphocyte antigen (e.g., B7-1, B7-3) or blocking antibody), prior to transplantation can lead to the binding of the molecule to the natural ligand(s) on the immune cells without transmitting the corresponding costimulatory signal. Blocking B lymphocyte antigen function in this matter prevents cytokine synthesis by immune cells, such as T cells, and thus acts as an immunosuppressant. Moreover, the lack of costimulation may also be sufficient to anergize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of a combination of B lymphocyte antigens.

The efficacy of particular blocking reagents in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow et al., Science 257:789-792 (1992) and Turka et al., Proc. Natl. Acad. Sci USA, 89:11102-11105 (1992). In addition, murine models of GVHD (see Paul ed., Fundamental Immunology, Raven Press, New York, 1989, pp. 846-847) can be used to determine the effect of blocking B lymphocyte antigen function in vivo on the development of that disease.

Blocking antigen function may also be therapeutically useful for treating autoimmune diseases. Many autoimmune disorders are the result of inappropriate activation of T cells that are reactive against self tissue and which promote the production of cytokines and autoantibodies involved in the pathology of the diseases. Preventing the activation of autoreactive T cells may reduce or eliminate disease symptoms. Administration of reagents which block costimulation of T cells by disrupting receptor:ligand interactions of B lymphocyte antigens can be used to inhibit T cell activation and prevent production of autoantibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well-characterized animal models of human autoimmune diseases. Examples include murine experimental autoimmune encephalitis, systemic lupus erythmatosis in MRL/lpr/lpr mice or NZB hybrid mice, murine autoimmune collagen arthritis, diabetes mellitus in NOD mice and BB rats, and murine experimental myasthenia gravis (see Paul ed., Fundamental Immunology, Raven Press, New York, 1989, pp. 840-856).

Upregulation of an antigen function (preferably a B lymphocyte antigen function), as a means of up regulating immune responses, may also be useful in therapy. Upregulation of immune responses may be in the form of enhancing an existing immune response or eliciting an initial immune response. For example, enhancing an immune response through stimulating B lymphocyte antigen function may be useful in cases of viral infection. In addition, systemic viral diseases such as influenza, the commoncold, and encephalitis might be alleviated by the administration of stimulatory forms of B lymphocyte antigens systemically.

Alternatively, anti-viral immune responses may be enhanced in an infected patient by removing T cells from the patient, costimulating the T cells in vitro with viral antigen-pulsed APCs either expressing a peptide of the present invention or together with a stimulatory form of a soluble peptide of the present invention and reintroducing the in vitro activated T cells into the patient. Another method of enhancing anti-viral immune responses would be to isolate infected cells from a patient, transfect them with a nucleic acid encoding a protein of the present invention as described herein such that the cells express all or a portion of the protein on their surface, and reintroduce the transfected cells into the patient. The infected cells would now be capable of delivering a costimulatory signal to, and thereby activate, T cells in vivo.

In another application, up regulation or enhancement of antigen function (preferably B lymphocyte antigen function) may be useful in the induction of tumor immunity. Tumor cells (e.g., sarcoma, melanoma, lymphoma, leukemia, neuroblastoma, carcinoma) transfected with a nucleic acid encoding at least one peptide of the present invention can be administered to a subject to overcome tumor-specific tolerance in the subject. If desired, the tumor cell can be transfected to express a combination of peptides. For example, tumor cells obtained from a patient can be transfected ex vivo with an expression vector directing the expression of a peptide having B7-2-like activity alone, or in conjunction with a peptide having B7-1-like activity and/or B7-3-like activity. The transfected tumor cells are returned to the patient to result in expression of the peptides on the surface of the transfected cell. Alternatively, gene therapy techniques can be used to target a tumor cell for transfection in vivo.

The presence of the peptide of the present invention having the activity of a B lymphocyte antigen(s) on the surface of the tumor cell provides the necessary costimulation signal to T cells to induce a T cell mediated immune response against the transfected tumor cells. In addition, tumor cells which lack MHC class I or MHC class II molecules, or which fail to reexpress sufficient amounts of MHC class I or MHC class II molecules, can be transfected with nucleic acid encoding all or a portion of (e.g., a cytoplasmic-domain truncated portion) of an MHC class I α chain protein and β₂ microglobulin protein or an MHC class IIα chain protein and an MHC class IIβ chain protein to thereby express MHC class I or MHC class II proteins on the cell surface. Expression of the appropriate class I or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a human subject may be sufficient to overcome tumor-specific tolerance in the subject.

The activity of a protein of the invention may, among other means, be measured by the following methods:

Suitable assays for thymocyte or splenocyte cytotoxicity include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Takai et al., J. Immunol. 140:508-512, 1988; Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Bowmanet al., J. Virology 61:1992-1998; Takai et al., J. Immunol. 140:508-512, 1988; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J. Immunol. 144:3028-3033, 1990; and Assays for B cell function: In vitro antibody production, Mond, J. J. and Brunswick, M. In Current Protocols in Immunology. J.E.e.a. Coligan eds. Vol 1 pp. 3.8.1-3.8.16, John Wiley and Sons, Toronto. 1994.

Mixed lymphocyte reaction (MLR) assays (which will identify, among others, proteins that generate predominantly Th1 and CTL responses) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Takai et al., J. Immunol. 140:508-512, 1988; Bertagnolli et al., J. Immunol. 149:3778-3783, 1992.

Dendritic cell-dependent assays (which will identify, among others, proteins expressed by dendritic cells that activate naive T-cells) include, without limitation, those described in: Guery et al., J. Immunol. 134:536-544, 1995; Inaba et al., Journal of Experimental Medicine 173:549-559, 1991; Macatonia et al., Journal of Immunology 154:5071-5079, 1995; Porgador et al., Journal of Experimental Medicine 182:255-260, 1995; Nair et al., Journal of Virology 67:4062-4069, 1993; Huang et al., Science 264:961-965, 1994; Macatonia et al., Journal of Experimental Medicine 169:1255-1264, 1989; Bhardwaj et al., Journal of Clinical Investigation 94:797-807, 1994; and Inaba et al., Journal of Experimental Medicine 172:631-640, 1990.

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, Journal of Immunology 145:4037-4045, 1990; Zamai et al., Cytometry 14:891-897, 1993; Gorczyca et al., International Journal of Oncology 1:639-648, 1992.

Assays for proteins that influence early steps of T-cell commitment and development include, without limitation, those described in: Antica et al., Blood 84:111-117, 1994; Fine et al., Cellular Immunology 155:111-122, 1994; Galy et al., Blood 85:2770-2778, 1995; Toki et al., Proc. Nat. Acad Sci. USA 88:7548-7551, 1991.

### Hematopoiesis Regulating Activity

A protein of the present invention may be useful in regulation of hematopoiesis and, consequently, in the treatment of myeloid or lymphoid cell deficiencies. Even marginal biological activity in support of colony forming cells or of factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional CSF activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantation, including, without limitation, aplastic anemia and paroxysmal nocturnal hemoglobinuria), as well as in repopulating the stem cell compartment post irradiation/chemotherapy, either in-vivo or ex-vivo (i.e., in conjunction with bone marrow transplantation or with peripheral progenitor cell transplantation (homologous or heterologous)) as normal cells or genetically manipulated for gene therapy.

The activity of a protein of the invention may, among other means, be measured by the following methods:

Suitable assays for proliferation and differentiation of various hematopoietic lines are cited above.

Assays for embryonic stem cell differentiation (which will identify, among others, proteins that influence embryonic differentiation hematopoiesis) include, without limitation, those described in: Johansson et al. Cellular Biology 15:141-151, 1995; Keller et al., Molecular and Cellular Biology 13:473-486, 1993; McClanahan et al., Blood 81:2903-2915, 1993.

Assays for stem cell survival and differentiation (which will identify, among others, proteins that regulate lympho-hematopoiesis) include, without limitation, those described in: Methylcellulose colony forming assays, Freshney, M. G. In Culture of Hematopoietic Cells. R. I. Freshney, et al. eds. Vol pp. 265-268, Wiley-Liss, Inc., New York, N.Y. 1994; Hirayama et al., Proc. Natl. Acad. Sci. USA 89:5907-5911, 1992; Primitive hematopoietic colony forming cells with high proliferative potential, McNiece, I. K. and Briddell, R. A. In Culture of Hematopoietic Cells. R. I. Freshney, et al. eds. Vol pp. 23-39, Wiley-Liss, Inc., New York, N.Y. 1994; Neben et al., Experimental Hematology 22:353-359, 1994; Cobblestone area forming cell assay, Ploemacher, R. E. In Culture of Hematopoietic Cells. R. I. Freshney, et al. eds. Vol pp. 1-21, Wiley-Liss, Inc., New York, N.Y. 1994; Long term bone marrow cultures in the presence of stromal cells, Spooncer, E., Dexter, M. and Allen, T. In Culture of Hematopoietic Cells. R. I. Freshney, et al. eds. Vol pp. 163-179, Wiley-Liss, Inc., New York, N.Y. 1994; Long term culture initiating cell assay, Sutherland, H. J. In Culture of Hematopoietic Cells. R. I. Freshney, et al. eds. Vol pp. 139-162, Wiley-Liss, Inc., New York, N.Y. 1994.

### Tissue Growth Activity

A protein of the present invention also may have utility in compositions used for bone, cartilage, tendon, ligament and/or nerve tissue growth or regeneration, as well as for wound healing and tissue repair and replacement, and in the treatment of burns, incisions and ulcers.

A protein of the present invention, which induces cartilage and/or bone growth in circumstances where bone is not normally formed, has application in the healing of bone fractures and cartilage damage or defects in humans and other animals. Such a preparation employing a protein of the invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery.

A protein of this invention may also be used in the treatment of periodontal disease, and in other tooth repair processes. Such agents may provide an environment to attract bone-forming cells, stimulate growth of bone-forming cells or induce differentiation of progenitors of bone-forming cells. A protein of the invention may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes.

Another category of tissue regeneration activity that may be attributable to the protein of the present invention is tendon/ligament formation. A protein of the present invention, which induces tendon/ligament-like tissue or other tissue formation in circumstances where such tissue is not normally formed, has application in the healing of tendon or ligament tears, deformities and other tendon or ligament defects in humans and other animals. Such a preparation employing a tendon/ligament-like tissue inducing protein may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the present invention contributes to the repair of congenital, trauma induced, or other tendon or ligament defects of other origin, and is also useful in cosmetic plastic surgery for attachment or repair of tendons or ligaments. The compositions of the present invention may provide an environment to attract tendon- or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions of the invention may also be useful in the treatment of tendinitis, carpal tunnel syndrome and other tendon or ligament defects. The compositions may also include an appropriate matrix and/or sequestering agent as a carrier as is well known in the art.

The protein of the present invention may also be useful for proliferation of neural cells and for regeneration of nerve and brain tissue, i.e. for the treatment of central and peripheral nervous system diseases and neuropathies, as well as mechanical and traumatic disorders, which involve degeneration, death or trauma to neural cells or nerve tissue. More specifically, a protein may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions which may be treated in accordance with the present invention include mechanical and traumatic disorders, such as spinal cord disorders, head trauma and cerebrovascular diseases such as stroke. Peripheral neuropathies resulting from chemotherapy or other medical therapies may also be treatable using a protein of the invention.

Proteins of the invention may also be useful to promote better or faster closure of non-healing wounds, including without limitation pressure ulcers, ulcers associated with vascular insufficiency, surgical and traumatic wounds, and the like.

It is expected that a protein of the present invention may also exhibit activity for generation or regeneration of other tissues, such as organs (including, for example, pancreas, liver, intestine, kidney, skin, endothelium), muscle (smooth, skeletal or cardiac) and vascular (including vascular endothelium) tissue, or for promoting the growth of cells comprising such tissues. Part of the desired effects may be by inhibition or modulation of fibrotic scarring to allow normal tissue to regenerate. A protein of the invention may also exhibit angiogenic activity.

A protein of the present invention may also be useful for gut protection or regeneration and treatment of lung or liver fibrosis, reperfusion injury in various tissues, and conditions resulting from systemic cytokine damage.

A protein of the present invention may also be useful for promoting or inhibiting differentiation of tissues described above from precursor tissues or cells; or for inhibiting the growth of tissues described above.

The activity of a protein of the invention may, among other means, be measured by the following methods:

Assays for tissue generation activity include, without limitation, those described in: International Patent Publication No. WO95/16035 (bone, cartilage, tendon); International Patent Publication No. WO95/05846 (nerve, neuronal); International Patent Publication No. WO91/07491 (skin, endothelium).

Assays for wound healing activity include, without limitation, those described in: Winter, Epidermal Wound Healing, pps. 71-112 (Maibach, H I and Rovee, D T, eds.), Year Book Medical Publishers, Inc., Chicago, as modified by Eaglstein and Mertz, J. Invest. Dermatol 71:382-84 (1978).

### Activin/Inhibin Activity

A protein of the present invention may also exhibit activin- or inhibin-related activities. Inhibins are characterized by their ability to inhibit the release of follicle stimulating hormone (FSH), while activins and are characterized by their ability to stimulate the release of follicle stimulating hormone (FSH). Thus, a protein of the present invention, alone or in heterodimers with a member of the inhibin α family, may be useful as a contraceptive based on the ability of inhibins to decrease fertility in female mammals and decrease spermatogenesis in male mammals. Administration of sufficient amounts of other inhibins can induce infertility in these mammals. Alternatively, the protein of the invention, as a homodimer or as a heterodimer with other protein subunits of the inhibin-A group, may be useful as a fertility inducing therapeutic, based upon the ability of activin molecules in stimulating FSH release from cells of the anterior pituitary. See, for example, U.S. Pat. No. 4,798,885. A protein of the invention may also be useful for advancement of the onset of fertility in sexually immature mammals, so as to increase the lifetime reproductive performance of domestic animals such as cows, sheep and pigs.

The activity of a protein of the invention may, among other means, be measured by the following methods:

Assays for activin/inhibin activity include, without limitation, those described in: Vale et al., Endocrinology 91:562-572, 1972; Ling et al., Nature 321:779-782, 1986; Vale et al., Nature 321:776-779, 1986; Mason et al., Nature 318:659-663, 1985; Forage et al., Proc. Natl. Acad. Sci. USA 83:3091-3095, 1986.

### Chemotactic/Chemokinetic Activity

A protein of the present invention may have chemotactic or chemokinetic activity (e.g., act as a chemokine) for mammalian cells, including, for example, monocytes, fibroblasts, neutrophils, T-cells, mast cells, eosinophils, epithelial and/or endothelial cells. Chemotactic and chemokinetic proteins can be used to mobilize or attract a desired cell population to a desired site of action. Chemotactic or chemokinetic proteins provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

A protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

The activity of a protein of the invention may, among other means, be measured by the following methods:

Assays for chemotactic activity (which will identify proteins that induce or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: Current Protocols in Immunology, Ed by J. E. Coligan, A. M. Kruisbeek, D. H. Margulies, E. M. Shevach, W.Strober, Pub. Greene Publishing Associates and Wiley-Interscience (Chapter 6.12, Measurement of alpha and beta Chemokines 6.12.1-6.12.28; Taub et al. J. Clin. Invest. 95:1370-1376, 1995; Lind et al. APMIS 103:140-146, 1995; Muller et al Eur. J. Immunol. 25: 1744-1748; Gruber et al. J. of Immunol. 152:5860-5867, 1994; Johnston et al. J. of Immunol. 153: 1762-1768, 1994.

### Hemostatic and Thrombolytic Activity

A protein of the invention may also exhibit hemostatic or thrombolytic activity. As a result, such a protein is expected to be useful in treatment of various coagulation disorders (includinghereditary disorders, such as hemophilias) or to enhance coagulation and other hemostatic events in treating wounds resulting from trauma, surgery or other causes. A protein of the invention may also be useful for dissolving or inhibiting formation of thromboses and for treatment and prevention of conditions resulting therefrom (such as, for example, infarction of cardiac and central nervous system vessels (e.g., stroke).

The activity of a protein of the invention may, among other means, be measured by the following methods:

Assay for hemostatic and thrombolytic activity include, without limitation, those described in: Linet et al., J. Clin. Pharmacol. 26:131-140, 1986; Burdick et al., Thrombosis Res. 45:413-419, 1987; Humphrey et al., Fibrinolysis 5:71-79 (1991); Schaub, Prostaglandins 35:467-474, 1988.

### Receptor/Ligand Activity

A protein of the present invention may also demonstrate activity as receptors, receptor ligands or inhibitors or agonists of receptor/ligand interactions. Examples of such receptors and ligands include, without limitation, cytokine receptors and their ligands, receptor kinases and their ligands, receptor phosphatases and their ligands, receptors involved in cell-cell interactions and their ligands (including without limitation, cellular adhesion molecules (such as selecting, integrins and their ligands) and receptor/ligand pairs involved in antigen presentation, antigen recognition and development of cellular and humoral immune responses). Receptors and ligands are also useful for screening of potential peptide or small molecule inhibitors of the relevant receptor/ligand interaction. A protein of the present invention (including, without limitation, fragments of receptors and ligands) may themselves be useful as inhibitors of receptor/ligand interactions.

The activity of a protein of the invention may, among other means, be measured by the following methods:

Suitable assays for receptor-ligand activity include without limitation those described in:Current Protocols in Immunology, Ed by J. E. Coligan, A. M. Kruisbeek, D. H. Margulies, E. M. Shevach, W.Strober, Pub. Greene Publishing Associates and Wiley-Interscience (Chapter 7.28, Measurement of Cellular Adhesion under static conditions 7.28.1-7.28.22), Takai et al., Proc. Natl. Acad. Sci. USA 84:6864-6868, 1987; Bierer et al., J. Exp. Med. 168:1145-1156, 1988; Rosenstein et al., J. Exp. Med. 169:149-160 1989; Stoltenborg et al., J. Immunol. Methods 175:59-68, 1994; Stitt et al., Cell 80:661-670, 1995.

### Anti-Inflammatory Activity

Proteins of the present invention may also exhibit anti-inflammatory activity. The anti-inflammatory activity may be achieved by providing a stimulus to cells involved in the inflammatory response, by inhibiting or promoting cell-cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Proteins exhibiting such activities can be used to treat inflammatory conditions including chronic or acute conditions), including without limitation inflammation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of ytokines such as TNF or IL-1. Proteins of the invention may also be useful to treat anaphylaxis and hypersensitivity to an antigenic substance or material.

### Tumor Inhibition Activity

In addition to the activities described above for immunological treatment or prevention of tumors, a protein of the invention may exhibit other anti-tumor activities. A protein may inhibit tumor growth directly or indirectly (such as, for example, via ADCC). A protein may exhibit its tumor inhibitory activity by acting on tumor tissue or tumor precursor tissue, by inhibiting formation of tissues necessary to support tumor growth (such as, for example, by inhibiting angiogenesis), by causing production of other factors, agents or cell types which inhibit tumor growth, or by suppressing, eliminating or inhibiting factors, agents or cell types which promote tumor growth

### Other Activities

A protein of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, or organ or body part size or shape (such as, for example, breast augmentation or diminution, change in bone form or shape); effecting biorhythms or caricadic cycles or rhythms; effecting the fertility of male or female subjects; effecting the metabolism, catabolism, anabolism, processing, utilization, storage or elimination of dietary fat, lipid, protein, carbohydrate, vitamins, minerals, cofactors or other nutritional factors or component(s); effecting behavioral characteristics, including, without limitation, appetite, libido, stress, cognition (including cognitive disorders), depression (including depressive disorders) and violent behaviors; providing analgesic effects or other pain reducing effects; promoting differentiation and growth of embryonic stem cells in lineages other than hematopoietic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

1. A protein containing any of the amino acid sequences represented by Sequence No. 1 to Sequence No. 2 or by Sequence No. 4 to Sequence No. 25.

2. A DNA encoding any of the proteins as described in claim 1.

3. A cDNA containing any of the base sequences represented by Sequence No. 26 to Sequence No. 50.

4. A cDNA as described in claim 3 which comprises any of the base sequences represented by Sequence No. 51 to Sequence No. 75.

5. A transformed eukaryotic cell capable of expressing any of DNAs as described in claim 2 to 4 and producing a protein as described in claim 1.
